(19) United States
(12) Patent Application Publication
(10) Pub. No.: US 2025/0144081 A1
(43) Pub. Date: May 8, 2025

(54) METHODS FOR REDUCING ALCOHOL, DRUG, AND FOOD INTAKE

(71) Applicant: The Scripps Research Institute, La Jolla, CA (US)

(72) Inventor: Pietro Paolo Sanna, San Diego, CA (US)

(21) Appl. No.: 18/835,631

(22) PCT Filed: Feb. 1, 2023
(86) PCT No.: PCT/US23/61778

§ 371 Date: Aug. 2, 2024

Related U.S. Application Data
(60) Provisional application No. 63/305,727, filed on Feb. 2, 2022.

(51) Int. Cl. A61K 31/4245; A61K 31/135; A61P 3/04; A61P 25/32

(52) U.S. Cl. CPC A61K 31/4245; A61K 31/135; A61P 3/04; A61P 25/32

(57) ABSTRACT

The present invention features methods for reducing alcohol intake, intake of drugs of abuse, food intake, or to increase fat breakdown or lipolysis by administering a sphingosine-1-phosphate receptor modulator.

### CROSS REFERENCE TO RELATED APPLICATION

This application claims priority to U.S. provisional patent application No. 63/305,727, which was filed on Feb. 2, 2022, and which is hereby incorporated by reference in its entirety.

### STATEMENT AS TO FEDERALLY FUNDED RESEARCH

This invention was made with government support under Grant Nos. AA025012, AA028982, AA021667, and DA046204 awarded by the National Institutes of Health. The government has certain rights in the invention.

### BACKGROUND OF THE INVENTION

In many countries, alcohol is the most prevalent abused substance. Alcohol use disorder (AUD) is the third-leading preventable cause of death in the US. An estimated 95,000 people die from alcohol-related causes annually. Few medications are available for AUD. In addition to alcohol, abused substances include stimulants like methamphetamine, cocaine, amphetamine, and methylphenidate; opioids such as heroin, morphine, fentanyl, oxycodone, hydrocodone, codeine, dihydrocodeine; nicotine; cannabinoids like tetrahydrocannabinol (THC); sedatives and hypnotics like barbiturates and benzodiazepines; psychedelics like lysergic acid diethylamide (LSD); anesthetics such as ketamine, ecstasy (MDMA); solvent drugs; designer drugs, among others. Few therapeutic options exist for drug abuse. Opioid use disorder is often treated with opioid replacement therapy using methadone or buprenorphine. However, no approved medications are currently available for stimulants like cocaine and methamphetamine and most other abused substances. Thus, the introduction of new safe and effective medications for substance use disorders is an unmet medical need.

Excessive alcohol intake results in multiple organ damage including brain, heart, liver and pancreas damage, delirium tremens, Wernicke-Korsakoff syndrome, increased prevalence of cancer, and liver disease. Alcoholic liver disease (ALD), also known as alcohol-related liver disease (ARLD), includes fatty liver, alcoholic hepatitis, and chronic hepatitis with liver fibrosis or cirrhosis. Drinking during pregnancy can result in fetal alcohol spectrum disorders. Excessive alcohol intake also leads to cognitive impairment and dementia. Complications of drug abuse include overdose, which can lead to death, HIV/AIDS, hepatitis C, problems meeting social or professional responsibilities, cardiovascular and nervous system toxicity, suicide, and cognitive impairments.

In addition to substance abuse, excessive eating and obesity are unmet medical needs. Feeding and eating disorders include binge eating disorder (BED); anorexia nervosa; bulimia nervosa, where individuals eat a large quantity (binging) then try to rid themselves of the food (purging); pica, where the patient eats non-food items; rumination syndrome, where the patient regurgitates undigested or minimally digested food; and avoidant/restrictive food intake disorder (ARFID). Compulsive eating behavior is associated with obesity and eating disorders. The biological bases of compulsive eating are still not well defined. Current understanding highlights three elements of compulsive behavior as it applies to pathological overeating: 1) habitual overeating; 2) overeating to relieve a negative emotional state; and 3) overeating despite aversive consequences. These elements emerge through mechanisms involving pathological habit formation through an aberrant learning process, the emergence of a negative emotional state, and dysfunctions in behavioral control. Dysfunctions in systems within neurocircuitries that comprise the basal ganglia, the extended amygdala, and the prefrontal cortex result in compulsive eating behaviors. There is an association between increased weight gain and increased inflammation.

Excessive eating can lead to obesity, diabetes, and increased prevalence of metabolic syndrome. Obesity is highly prevalent in western societies and is associated with metabolic dysregulations, dyslipidemia, insulin resistance, hyperglycemia, diabetic neuropathy, diabetic nephropathy, diabetic retinopathy, nonalcoholic fatty liver disease (NAFLD), hypertension, and cardiovascular disease. Obesity and type 2 diabetes are risk factors for NAFLD. NAFLD, also referred to as metabolic (dysfunction) associated fatty liver disease (MAFLD), is due to excessive fat accumulation in the liver, or steatosis, and can progress to non-alcoholic steatohepatitis (NASH), fibrosis, and cirrhosis and is a risk factor for hepatocellular carcinoma. Substance use and misuse may be associated with eating disorders, either preceding or following or can be co-occurring. Individuals with eating disorders are more likely to use alcohol or illicit drugs.

Thus, the development of more efficacious and safe therapeutic options for treating or preventing substance (e.g., alcohol and drug) abuse and dependence and eating disorders and obesity are needed.

### SUMMARY OF THE INVENTION

In one aspect, the invention features a method of reducing excessive alcohol intake, intake of a drug of abuse, or food in a subject by administering a pharmaceutical composition that includes a sphingosine-1 phosphate (S1P) receptor (S1PR) modulator in an amount and for a duration sufficient to reduce alcohol intake, intake of a drug of abuse, or food intake. The S1PR modulator may be, for example, an S1PR1, S1PR2, S1PR3, S1PR4, and/or an S1PR5 modulator. The S1PR modulator may be, e.g., a compound selected from Table 1.

In some embodiments, the method reduces alcohol intake. In some embodiments, the method reduces excessive alcohol intake.

In some embodiments, the method reduces intake of a drug of abuse. In some embodiments, the method reduces excessive intake of a drug of abuse.

In some embodiments, the method reduces eating. In some embodiments, the method reduces excessive feeding or eating (e.g., binge eating).

In some embodiments, the method reduces co-occurring substance abuse and eating disorder.

In some embodiments, the method increases fat utilization, e.g., fat burning or fat breakdown, also known as lipolysis or fat oxidation or β-oxidation.

In some embodiments, the method reduces inflammation.

In some embodiments, the pharmaceutical composition suppresses dependence or craving for alcohol, drugs of abuse, or food in the subject.

In some embodiments, the pharmaceutical composition is administered orally.

In some embodiments, the subject suffers from or has previously suffered from alcoholism, an alcohol use disorder (AUD), alcohol abuse, relapse to excessive drinking, alcohol dependence, drug abuse, substance use disorder (SUD), drug dependence, relapse to drug taking, an eating disorder or obesity or is overweight.

In some embodiments, the eating disorder is binge eating disorder (BED), anorexia nervosa, bulimia nervosa, pica, rumination syndrome, avoidant/restrictive food intake disorder (ARFID), or compulsive eating behavior.

In some embodiments, the method results in weight loss or fat loss.

In some embodiments, the S1PR modulator is an agonist.

In some embodiments, the agonist is fingolimod, ozanimod (RPC1063), siponimod (BAF312), ponesimod (ACT128800), A971432, CYM-5442, S1P, TC-SP 14, SEW 2871, RP 001 hydrochloride, CS 2100, RP 001, ASP4058, AMG 369, AUY954, KRP-203, Ceralifimod (ONO-4641), GSK2018682, CS-0777, VPC22277, carbamoyl-nicotinamide, laquinimod, cenerimod, AKP11, or a pharmaceutically acceptable salt thereof.

In some embodiments, the pharmaceutical composition includes fingolimod.

In some embodiments, the pharmaceutical composition includes ozanimod.

In some embodiments, the pharmaceutical composition includes CYM-5442.

In some embodiments, the pharmaceutical composition includes A971432.

In some embodiments, the pharmaceutical composition includes siponimod.

In some embodiments, the S1PR modulator is an antagonist.

In some embodiments, the antagonist is CAY10444, etrasimod, amiselimod (MT-1303), W146, VPC 23019, JTE 013, AB1, CYM 50260, CYM 50308, AAL-R, VPC03090, VPC44116, NIBR-0213, or a pharmaceutically acceptable salt thereof.

In some embodiments, the pharmaceutical composition includes CAY10444.

### Definitions

As used herein, the term “about” means+/−10% of the recited value.

As used herein, by “administering” is meant a method of giving a dosage of a pharmaceutical composition (e.g., a small molecule or an antibody or antigen-binding fragment thereof as described herein or any of the other agents described herein). The compositions utilized in the methods described herein can be administered, for example, orally, intravenously, subcutaneously, subconjunctivally, intravesicularlly, mucosally, intrapericardially, intraumbilically, intraocularly, intraperitoneally, topically, locally, by inhalation, by injection, by infusion, by continuous infusion, by localized perfusion bathing target cells directly, by catheter, by lavage, by gavage, in cremes, or in lipid compositions, according to methods known in the art for the particular agent. The method of administration can vary depending on various factors (e.g., the components of the composition that is administered and the severity of the condition being treated) and by using known guidance for the agent being administered.

By “pharmaceutical composition” is meant any composition that contains a therapeutically or biologically active agent, such as a small molecule or an antibody or antigen-binding fragment thereof as described herein or any of the other agents described herein. For the purposes of this disclosure, pharmaceutical compositions include the active agent and one or more pharmaceutically acceptable carriers, excipients, or diluents known in the art to be suitable for delivering a therapeutic or biologically active agent.

As used herein, “treatment” is an approach for obtaining beneficial or desired results, such as clinical results. Beneficial or desired results can include, but are not limited to, alleviation or amelioration of one or more symptoms of alcoholism, an alcohol use disorder, alcohol abuse, or alcohol dependence, substance use disorder. A treatment can include one or more therapeutic agents, such as one or more of the compositions described herein and/or one or more additional therapeutic agents.

As used herein, “alcohol use disorder” or “AUD” describes a medical condition characterized by an impaired ability to stop or control alcohol consumption despite the negative social, occupational or health consequences.

As used herein, “substance use disorder or “SUD” is the persistent use of drugs (including alcohol) despite substantial harm and adverse consequences as a result of their use. Substance use disorder is a cluster of cognitive, behavioral, and physiological symptoms indicating that the individual continues using the substance despite significant substance-related problems, including impaired control, social impairment, and risky use. These substances can include alcohol; caffeine; cannabis; hallucinogens (with separate categories for phencyclidine (or similarly acting arylcyclohexylamines and other hallucinogens); inhalants; opioids; sedatives, hypnotics, or anxiolytics; stimulants (amphetamine-type substances, cocaine, and other stimulants); tobacco (nicotine); and other (or unknown) substances.

As used herein, “feeding and eating disorders” are mental disorders defined by abnormal eating behaviors that negatively affect a person's physical or mental health. Feeding and eating disorders are characterized by a persistent disturbance of eating or eating-related behavior that results in the altered consumption or absorption of food and that significantly impairs physical health or psychosocial functioning. Diagnostic criteria are provided for pica, rumination disorder, avoidant/restrictive food intake disorder, anorexia nervosa, bulimia nervosa, and binge-eating disorder.

### BRIEF DESCRIPTION OF THE DRAWINGS

FIGS. 1A and 1B are graphs showing that the sphingosine-1-phosphate (S1P) receptor agonist ozanimod reduces alcohol intake in the drinking in the dark (DID) binge drinking paradigm.

FIG. 1A shows that ozanimod (0.25-1 mg/kg), by intraperitoneal administration (i.p.) reduced alcohol consumption in the DID paradigm after 2 h in C57BL/6J mice (F_{(3, 43)}=2.98; p<0.05; *p<0.05 by Fisher LSD post hoc). FIG. 1B shows that the reducing effect of ozanimod was not evident at the end of the 4-h drinking session (F_{(3, 43)}=2.109, p>0.05), which is consistent with its short half-life (172). Bars represent the mean±SEM of n=11-12 mice.

FIGS. 2A and 2B are graphs showing that the agonist fingolimod (FTY-720) reduces alcohol intake in the DID binge drinking paradigm. Fingolimod (4 mg/kg, i.p.) reduced alcohol consumption in the DID paradigm over a 4 h drinking session in C57BL/6J mice (FIG. 2A shows first 2 h: 1-way ANOVA F(3, 52)=7.62, p<0.0005; FIG. 2B shows 4 h: F(3, 52)=9.87, p<0.0001; ****p<0.0001 by Fisher LSD post hoc). Bars represent the mean±SEM of n=13-15 mice.

FIGS. 3A and 3B are graphs showing that the S1P receptor agonist ozanimod increases alcohol sensitivity. FIG. 3A shows ozanimod (0.1 and 0.3 mg/kg, i.p.) significantly increased the duration of loss of righting reflex (LORR), a measure of sensitivity to alcohol's hypnotic action, in C57BL/6J mice (F_{(2, 15)}=3.752; p<0.05; *p<0.05 by Fisher LSD post hoc). FIG. 3B shows blood alcohol levels (BALs) measured 70 min after alcohol injection were not affected by administration of ozanimod (F_{(2, 15)}=0.07, p>0.05). Bars represent the mean±SEM of n=6 mice.

FIGS. 4A-4E are graphs showing that the sphingosine kinase (SphK) inhibitor PF-543 increased alcohol intake in low drinking MeCP2^{308/Y} mice in the DID paradigm. FIGS. 4A-4C show that MeCP2^{308/Y} vehicle-treated mice consumed less alcohol than wild-type (WT) vehicle-treated mice in the first two hours (FIG. 4A) of the DID paradigm (*p<0.05) and over the 4 h session (**p<0.05) (FIG. 4C). Also shown is the last two hours (FIG. 4B). Administration of PF-543 (10 mg/kg, i.p.), 30 min before the start of the drinking session, increased alcohol consumption of MeCP2^{308/Y} mice, which was not different from the WT drug-treated mouse group (p>0.05). FIGS. 4D and 4E show that BALs were measured at the end of the 4 h drinking session in WT (FIG. 4D) and MeCP2^{308/Y} mice (FIG. 4E). The increased alcohol consumption of MeCP2^{308/Y} mice induced by administration of PF-543 was also reflected in higher BALs (p=0.06).

FIGS. 5A-5D are graphs showing that S1P receptor 1 agonist CYM-5442 reduces alcohol intake in male C57BL/6J mice in the DID binge drinking paradigm and saccharine in the same paradigm without affecting motor coordination in the rotarod test. FIG. 5A are graphs showing that CYM-5442 (2.5, 5 mg/kg, i.p.) reduces alcohol consumption in the DID paradigm over a 4 h drinking session in male C57BL/6J mice (first 2 h: 1-way ANOVA F_{(3, 36)}=13.66, p<0.0001; 1-way ANOVA 4 h: F_{(3, 36)}=17.46, p<0.0001; *p<0.05; **p<0.005 ****p<0.0001 by Tukey's post hoc). Bars represent the mean±SEM of n=10 mice. FIG. 5B are graphs showing that CYM-5442 (5 mg/kg, i.p.) reduces saccharin [0.002% (w/v)] consumption in the DID paradigm over a 4 h drinking session in male C57BL/6J mice (1-way ANOVA first 2 h: 1-way ANOVA F_{(3, 31)}=4.5, p<0.01; 1-way ANOVA 4 h: F_{(3, 31)}=4.88, p<0.01; **p<0.05 by Tukey's post hoc). Bars represent the mean±SEM of n=8-9 mice. On the Left panel, that CYM-5442 (2.5 mg/kg, i.p.) significantly increases the duration of loss of righting reflex (LORR) (3.5 g/kg), a measure of sensitivity to alcohol's hypnotic action, in male C57BL/6J mice (t=2.83, df=13; p<0.05; Unpaired T-test). Bars represent the mean±SEM of n=7-8 mice. FIG. 5C are graphs showing that CYM-5442 does not alter motor coordination performance of male C57BL/6J mice in the rotarod apparatus. 1-way ANOVA Latency to fall: F_{(3, 44)}=0.11, p>0.05; 1-way ANOVA Active run time: F_{(3, 44)}=0.14, p>0.05. Bars represent the mean±SEM of n=12 mice. FIG. 5D is a graph showing that CYM-5442 (2.5 mg/kg, i.p.) does not alter alcohol metabolism. Shown are blood alcohol levels (BALs) measured after alcohol injection (3.5 g/kg) in male C57BL/6J mice. 2-way repeated measures (RM) ANOVA, Time F_{(3, 42)}=1159, p<0.0001; Treatment F_{(1, 14)}=5.60, p<0.05; Interaction F_{(3, 42)}=0.15, p>0.05. Points represent the mean±SEM of n=7-8 mice.

FIGS. 6A-6C are graphs showing that the S1P receptor agonist CYM-5442 decreases intravenous methamphetamine self-administration in Wistar rats without producing motor impairment at the dose of 5 mg/kg. FIG. 6A is a graph showing that CYM-5442 decreases intravenous methamphetamine self-administration under a fixed ratio schedule of reinforcement in male Wistar rats with implanted chronic Micro-Renathane catheters. Rats were trained to self-administer methamphetamine under a fixed-ratio 1 schedule of reinforcement (FR1) whereby one lever press results in the delivery of one methamphetamine infusion (0.05 mg/kg) under 1-h daily sessions. Once lever-responding stabilized, rats were divided in two groups and treated either with vehicle or CYM-5442. Acute administration of CYM-5442 (5 mg/kg, i.p.) reduced lever-responding for methamphetamine in comparison to the vehicle-treated rat group over 1-h SA session (Unpaired T-test, t=2.21, df=14; *p<0.05). FIG. 6B is a graph showing that CYM-5442 decreases intravenous methamphetamine self-administration under a progressive ratio schedule of reinforcement in male Wistar rats with implanted chronic Micro-Renathane catheters. Following 2 weeks of methamphetamine self-administration under FR1, two balanced groups of rats were injected with either vehicle or CYM-5442 and tested under a Progressive Ratio (PR) schedule of reinforcement in which the response requirement was increased progressively. CYM-5442-treated rats had less motivation for methamphetamine as compared to the vehicle-treated rat as indicated by lower break point value (Unpaired T-test, t=2.35, df=11; *p<0.05). FIG. 6C is a graph showing that CYM-5442 did not affect motor coordination of an independent group of male Wistar rats tested in the rotarod apparatus (Unpaired T-test, t=2.35, df=11; *p<0.05).

FIGS. 7A and 7B are graphs showing that food-intake is reduced by administration of CYM-5442 in dose-dependent manner. FIG. 7A is a graph showing that food-intake during the 1^{st} 2-hrs was significantly reduced by 2.5 mg/kg and 5 mg/kg of CYM-5442 administration (One-way ANOVA, F(3,26)=7.886, P=0.0007, *p<0.05,**p<0.01 vs 0 mg/kg, Tukey's post hoc test); FIG. 7B is a graph showing that food-intake was reduced over the entire 4-hrs session by 5 mg/kg of CYM-5442 administration (One-way ANOVA, F(3,26)=3.014, P=0.0481, *p<0.05, vs 0 mg/kg, Tukey's post hoc test).

FIGS. 8A and 8B are graphs showing that CYM-5442 administration reduces Respiratory Exchange Ratio (RER). FIG. 8A is a graph showing that RER was significantly reduced by the administration of CYM-5442 (i.p.) for 1-3 hours in a dose-dependent manner. Repeated two-way ANOVA revealed a significant main effect of Time (F(82,2131)=35.66, P<0.0001) and significant interaction of Time×Dose (F(246,2131)=1.934, P<0.0001). *p<0.05, 0 mg/kg vs 5 mg/Kg; #p<0.05, 0 mg/kg vs 2.5 mg/Kg; $ #p<0.05, 0 mg/kg vs 1.25 mg/kg, respectively, Tukey's Post hoc test, n=7-8. FIG. 8A is a graph showing the average of 2 hours of RER indicating that CYM-5442 (i.p.) significantly reduced RER at the first 2 hrs measure point (1-3 hrs after injection). Two-way RM ANOVA revealed a significant main effect of Time (F(1.5, 39)=34.76, P<0.0001) and significant interaction of Time×Dose (F(6,52)=6.033, P<0.0001). *p<0.05, 0 mg/kg vs 5 mg/kg, Tukey's Post hoc test, n=7-8.

FIGS. 9A-9C are graphs showing the effect of chronic treatment with CYM-5442 on body weight, food and water intake in male C57Bl/6J mice. FIG. 9A is a graph showing the body weights of C57BL/6J mice (N=11-12/group) fed either with a low-fat diet (LFD) or a high-fat diet (HFD) and treated daily with CYM-5442 (2.5 mg/kg, i.p.) or vehicle 5 days a week over 7 consecutive weeks. CYM-5442 effectively prevented body weight gain in HFD-fed mice in comparison to vehicle-treated HFD-fed mice and the LFD-fed groups (3 way RM ANOVA: Time F(7, 301)=63.57; p<0.0001; Diet F(1, 43)=21.43), p<0.0001; Treatment (1, 43)=4.66, p<0.05; Time×Diet (7, 301)=14.47; p<0.0001; Time×Treatment (7, 301)=8.63; p<0.0001; Diet×Treatment (1, 43)=1.09, p>0.05; Time×Diet×Treatment (7, 301)=1.82, p>0.05. *p<0.05, **p<0.005, ****p<0.0001 by Fisher LSD post hoc). FIGS. 9B-C are graphs showing that the overall daily (24 h) food (g/kg) and water intake (ml/kg), did not differ between vehicle/CYM-5442-treated mice in either HFD-fed or LFD-fed mice (Food intake 3-way RM ANOVA: Time F(7, 300)=146.8; p<0.0001; Diet F(1, 43)=18.61), p<0.0001; Treatment (1, 43)=0.04, p>0.05; Time×Diet (7, 300)=29.26; p<0.0001; Time×Treatment (7, 300)=0.32; p>0.05; Diet×Treatment (1, 43)=3.58, p>0.05; Time×Diet×Treatment (7, 300)=1.45, p>0.05; Water intake 3 way RM ANOVA: Time F(7, 299)=180.3; p<0.0001; Diet F(1, 43)=29.12), p<0.0001; Treatment (1, 43)=0.02, p>0.05; Time×Diet (7, 299)=3.73; p<0.0001; Time×Treatment (7, 299)=0.96; p>0.05; Diet×Treatment (1, 43)=1.35, p>0.05; Time×Diet×Treatment (7, 299)=1.39, p>0.05).

FIGS. 10A and 10B are graphs showing that chronic CYM-5442 administration selectively reduces body fat in mice chronically fed a high-fat diet (HFD) without affecting lean mass. FIG. 10A is a graph showing that body fat was significantly increased by HFD diet. Chronic CYM-5442 treatment (2.5 mg/kg, i.p.) significantly reduced body fat in the HFD-fed group but not in a low-fat diet (LFD)-fed control group. 2-way ANOVA revealed a significant main effect of Diet (F1, 43=147.7, p<0.0001) and Treatment (F1, 43=16.13, p=0.0002), and a significant interaction of Diet and Treatment (F1, 43=16.70, p=0.0002). (****, p<0.0001, vs Vehicle in HFD, Turkey's post hoc test, n=11-12). FIG. 10B is a graph showing that lean Body Mass was affected by neither Diet (F1, 43=0.646, n.s.) nor by CYM-5442 treatment (F1, 43=0.00003359, n.s.). Body composition was determined by quantitative nuclear magnetic resonance (qNMR).

### DETAILED DESCRIPTION

The present invention features methods for reducing alcohol, drug, or food intake and increasing fat breakdown. The present invention is based in part on the surprising discovery that sphingosine-1-phosphate (S1P) signaling is a regulator of alcohol sensitivity and intake. The present invention is also premised on the discovery that S1P signaling is a modulator of food and drug intake. S1P is a lipid mediator that is generated from phosphorylation of sphingosine by the action of sphingosine kinase (SphK) isoenzymes in the brain and other tissues. S1P acts on five G protein-coupled receptors (S1P1-5). Accordingly, by targeting one or more of these receptors, a subject can be treated to reduce alcohol intake (e.g., excessive alcohol intake) and/or food intake (e.g., excessive food intake).

### EMBODIMENTS

Embodiment 1. A method of reducing alcohol intake, intake of a drug of abuse, or food intake in a subject comprising administering a pharmaceutical composition comprising a sphingosine-1 phosphate (S1P) receptor (S1PR) modulator in an amount and for a duration sufficient to reduce alcohol intake, intake of a drug of abuse, or food intake.

Embodiment 2. The method of embodiment 1, wherein the method reduces alcohol intake.

Embodiment 3. The method of embodiment 1 or 2, wherein the S1PR modulator is an S1PR1, S1PR2, S1PR3, S1PR4, and/or an S1PR5 modulator.

Embodiment 4. The method of any one of embodiments 1-3, wherein the pharmaceutical composition suppresses dependence or craving for alcohol or a drug of abuse or food in the subject.

Embodiment 5. The method of any one of embodiments 1-4, wherein the pharmaceutical composition is administered orally.

Embodiment 6. The method of any one of embodiments 1-5, wherein the subject suffers from or has previously suffered from alcoholism, an alcohol use disorder, alcohol abuse, alcohol dependence, or relapse to excessive drinking.

Embodiment 7. The method of any one of embodiments 1-6, wherein the subject suffers from or has previously suffered from drug abuse, substance abuse, substance misuse, or relapse to drug taking.

Embodiment 8. The method of any one of embodiments 1-7, wherein the subject suffers from or has previously suffered from an eating disorder or obesity or is overweight.

Embodiment 9. The method of embodiment 8, wherein the eating disorder is binge eating disorder, anorexia nervosa, bulimia nervosa, pica, rumination syndrome, avoidant/restrictive food intake disorder, or compulsive eating behavior.

Embodiment 10. The method of any one of embodiments 1-9, wherein the method results in weight loss, increased fat breakdown or fat oxidation, or fat loss or lipolysis.

Embodiment 11. The method of any one of embodiments 1-10, wherein the method reduces inflammation.

Embodiment 12. The method of any one of embodiments 1-11, wherein the S1PR modulator is an agonist.

Embodiment 13. The method of any one of embodiments 1-12, wherein the agonist is fingolimod, ozanimod, siponimod, ponesimod, A971432, CYM-5442, S1P, TC-SP 14, SEW2871, RP 001 hydrochloride, CS 2100, RP 001, ASP4058, AMG 369, AUY954, KRP-203, ceralifimod, GSK2018682, CS-0777, VPC22277, carbamoyl-nicotinamide, or a pharmaceutically acceptable salt thereof.

Embodiment 14. The method of embodiment 13, wherein the pharmaceutical composition comprises fingolimod.

Embodiment 15. The method of embodiment 13, wherein the pharmaceutical composition comprises ozanimod.

Embodiment 16. The method of embodiment 13, wherein the pharmaceutical composition comprises CYM-5442.

Embodiment 17. The method of embodiment 13, wherein the pharmaceutical composition comprises A971432.

Embodiment 18. The method of embodiment 13, wherein the pharmaceutical composition comprises siponimod.

Embodiment 19. The method of any one of embodiments 1-11, wherein the S1PR modulator is an antagonist.

Embodiment 20. The method of embodiment 19, wherein the antagonist is CAY10444, etrasimod, amiselimod (MT-1303), W146, VPC 23019, JTE 013, AB1, CYM 50260, CYM 50308, AAL-R, VPC03090, VPC44116, NIBR-0213, or a pharmaceutically acceptable salt thereof.

Embodiment 21. The method of embodiment 20, wherein the pharmaceutical composition comprises CAY10444.

Embodiment 22. The method of any one of embodiments 1-11, wherein the S1PR modulator is a compound selected from Table 1. The application provides that the reference to “a compound” in Embodiment 22 is intended refer to inclusively each species compound listed in Table 1 as provided hereinbelow.

The methods described herein include administering an S1PR modulator to a subject in an amount and for a duration sufficient to reduce intake (e.g., excessive intake) of alcohol, drug, or food and increasing fat breakdown as indicated by reduced respiratory exchange ratio (RER). The subject may suffer from or have previously suffered from alcoholism, an alcohol use disorder, alcohol abuse, or dependence. The subject may be a binge drinker. The subject may be an impulsive drinker. The subject may have increased dependence or cravings for alcohol. The methods may reduce or suppress (e.g., by at least 10%, 20%, 30%, 40%, 50%, 60%, 70%, 80%, 90%, 95%, 97%, or 100%) dependence or craving for alcohol, drugs of abuse, or food (e.g., as compared to a control subject or the subject before suffering from dependence or craving for alcohol, drugs of abuse, or food).

In some embodiments, the drug of abuse is methamphetamine, cocaine, amphetamine, methylphenidate, opioids such as heroin, morphine, fentanyl, oxycodone, hydrocodone, codeine or dihydrocodeine, nicotine, cannabinoids such as tetrahydrocannabinol (THC), depressants such as barbiturates and benzodiazepines, psychedelics (e.g., LSD), anesthetics (e.g., ketamine), ecstasy (MDMA), solvent drugs, designer drugs, or others.

In some embodiments, the method reduces eating. In some embodiments, the method reduces excessive eating (e.g., binge eating). In some embodiments, the method improves metabolism and increases fat oxidation. In some embodiments, the method reduces inflammation. In some embodiments, the pharmaceutical composition suppresses dependence or craving for alcohol, drugs of abuse, or food in the subject. In some embodiments, the method reduces co-occurring substance abuse and eating disorder.

In some embodiments, the subject suffers from or has previously suffered from alcoholism, an alcohol use disorder, alcohol abuse, alcohol dependence, relapse to excessive drinking, drug abuse, relapse to drug use, an eating disorder or obesity or is overweight. In some embodiments, the eating disorder is binge eating disorder (BED), anorexia nervosa, bulimia nervosa, pica, rumination syndrome, avoidant/restrictive food intake disorder (ARFID), or compulsive eating behavior.

Carbohydrates and fat are the main fuel sources that the body utilizes to produce energy. Through aerobic metabolism, oxygen consumption provides energy and carbon dioxide (CO₂) as a byproduct. The respiratory exchange ratio (RER), also referred to as the respiratory quotient (RQ), is a measure of the amount of the CO₂ produced divided by the amount of oxygen consumed (VO₂). The RER allows one to determine the predominant type of fuel utilized by the body whether carbohydrates or fat. A decrease in RER reflects the reduction in lipid synthesis and increased fat oxidation (β-oxidation) and lipolysis mechanisms and an increase in fat breakdown for energy use. Fatty acid β-oxidation is the process by which fatty acids are broken down to produce energy. Low-carbohydrate, high-fat (LCHF) diets result in higher rates of fat oxidation. A ketogenic diet (KD) is a nutritional approach that may be adopted for weight loss and restricts daily carbohydrates, thus resulting in a decreased RER and increased fat breakdown. In physically active individuals, KD results in loss of fat mass without any detrimental effects on strength, power, and muscle mass. Thus, a reduction of RER can be a tool to promote fat loss in the setting of physical activity and caloric intake reduction.

The S1PR modulator may be, for example an S1PR1, S1PR2, S1PR3, S1PR4, and/or an S1PR5 modulator. In some embodiments, the S1PR modulator is an S1PR1, S1PR2, S1PR3, S1PR4, and an S1PR5 modulator (e.g., fingolimod). In some embodiments, the S1PR modulator is an S1PR1 and an S1P5 modulator (e.g., ozanimod). In some embodiments, the S1PR modulator is an S1PR1 modulator (e.g., CYM-5442 or ponesimod, e.g., CYM-5442). In some embodiments, the S1PR modulator is an agonist. Alternatively, the S1PR modulator may be an antagonist. The modulator may be a small molecule or a polypeptide. The polypeptide may be, for example, an antibody or antigen-binding fragment thereof. In some embodiments, the S1PR modulator is a compound shown in Table 1.

TABLE 1
S1PR modulators

| Compound | Compound | Agonist or | |
|---|---|---|---|
| Number | Name | Antagonist | Structure |
| 1 | fingolimod | Agonist | 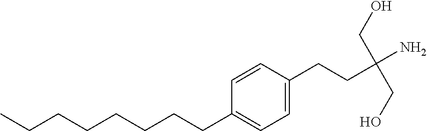 [SMILES: CCCCCCCCc1ccc(CCC(N)(CO)CO)cc1] |
| 2 | Ozanimod (RPC1063) | Agonist | 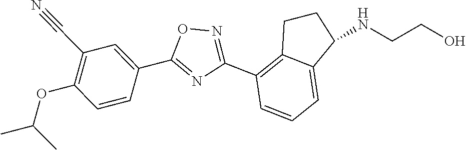 [SMILES: CC(C)Oc1ccc(-c2nc(-c3cccc4c3CC[C@@H]4NCCO)no2)cc1C#N] |
| 3 | 11 iponimod (BAF312) | Agonist | 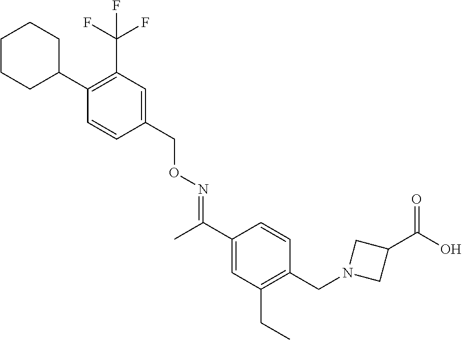 [SMILES: CCc1cc(/C(C)=N/OCc2ccc(C3CCCCC3)c(C(F)(F)F)c2)ccc1CN1CC(C(=O)O)C1] |
| 4 | Ponesimod (ACT128800) | Agonist | 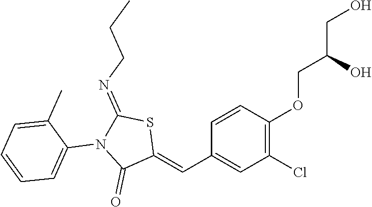 [SMILES: CCC/N=C1\S/C(=C\c2ccc(OC[C@H](O)CO)c(Cl)c2)C(=O)N1c1ccccc1C] |
| 5 | A971432 | Agonist | 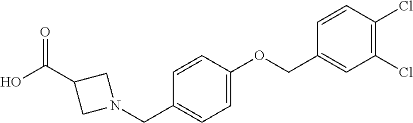 [SMILES: O=C(O)C1CN(Cc2ccc(OCc3ccc(Cl)c(Cl)c3)cc2)C1] |
| 6 | BMS-986166 Phosphate | Agonist | 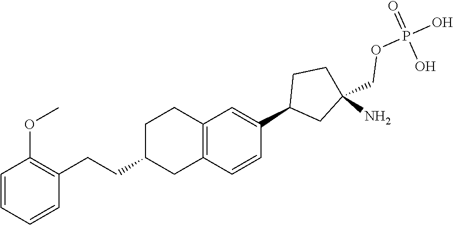 [SMILES: COc1ccccc1CC[C@@H]1CCc2cc([C@H]3CC[C@](N)(COP(=O)(O)O)C3)ccc2C1] |
| 7 | CYM-5442 | Agonist | 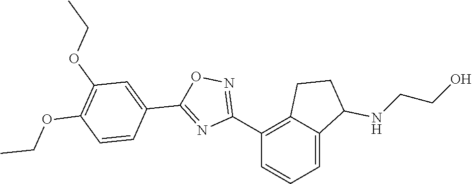 [SMILES: CCOc1ccc(-c2nc(-c3cccc4c3CCC4NCCO)no2)cc1OCC] |
| 8 | S1P | Agonist | 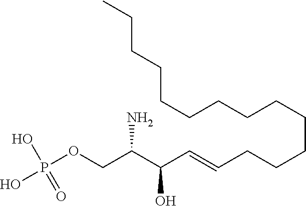 [SMILES: CCCCCCCCCCCCC/C=C/[C@@H](O)[C@@H](N)COP(=O)(O)O] |
| 9 | TC-SP 14 | Agonist | 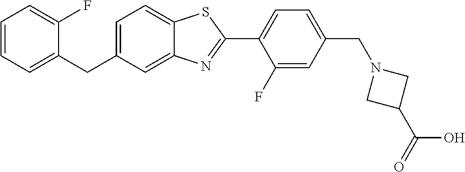 [SMILES: O=C(O)C1CN(Cc2ccc(-c3nc4cc(Cc5ccccc5F)ccc4s3)c(F)c2)C1] |
| 10 | SEW 2871 | Agonist | 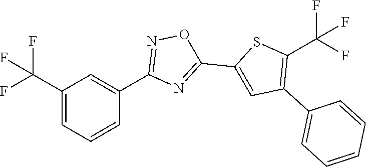 [SMILES: FC(F)(F)c1cccc(-c2noc(-c3cc(-c4ccccc4)c(C(F)(F)F)s3)n2)c1] |
| 11 | RP 001 hydrochloride | Agonist | 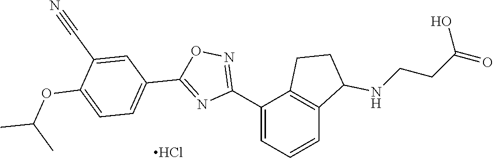 [SMILES: CC(C)Oc1ccc(-c2nc(-c3cccc4c3CCC4NCCC(=O)O)no2)cc1C#N ; Cl] |
| 12 | CS 2100 | Agonist | 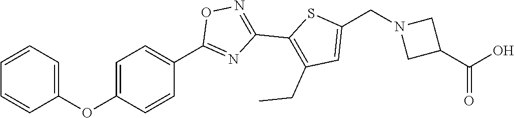 [SMILES: CCc1cc(CN2CC(C(=O)O)C2)sc1-c1noc(-c2ccc(Oc3ccccc3)cc2)n1] |
| 13 | RP 001 | Agonist | 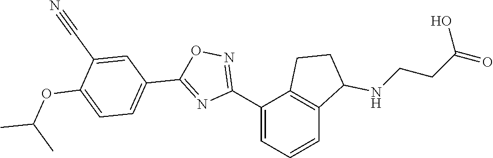 [SMILES: CC(C)Oc1ccc(-c2nc(-c3cccc4c3CCC4NCCC(=O)O)no2)cc1C#N] |
| 14 | ASP4058 | Agonist | 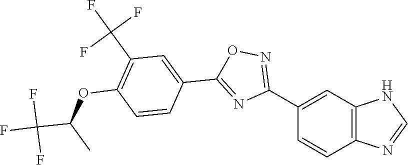 [SMILES: C[C@H](Oc1ccc(-c2nc(-c3ccc4nc[nH]c4c3)no2)cc1C(F)(F)F)C(F)(F)F] |
| 15 | AMG 369 | Agonist | 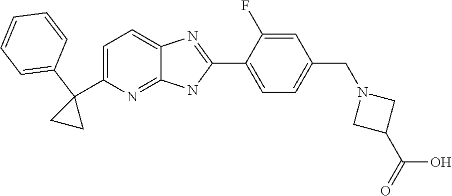 [SMILES: O=C(O)C1CN(Cc2ccc(-c3nc4ccc(C5(c6ccccc6)CC5)nc4[nH]3)c(F)c2)C1] |
| 16 | AUY954 | Agonist | 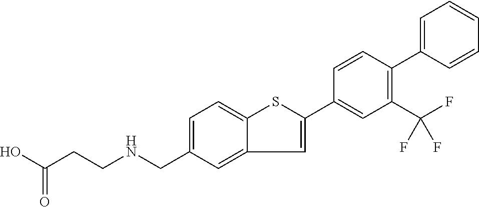 [SMILES: O=C(O)CCNCc1ccc2sc(-c3ccc(-c4ccccc4)c(C(F)(F)F)c3)cc2c1] |
| 17 | KRP-203 | Agonist | 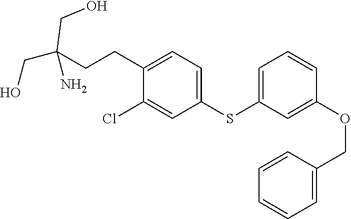 [SMILES: NC(CO)(CO)CCc1ccc(Sc2cccc(OCc3ccccc3)c2)cc1Cl] |
| 18 | Ceralifimod (ONO-4641) | Agonist | 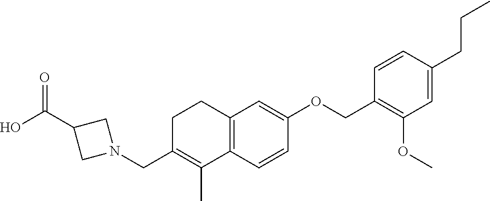 [SMILES: CCCc1ccc(COc2ccc3c(c2)CCC(CN2CC(C(=O)O)C2)=C3C)c(OC)c1] |
| 19 | GSK2018682 | Agonist | 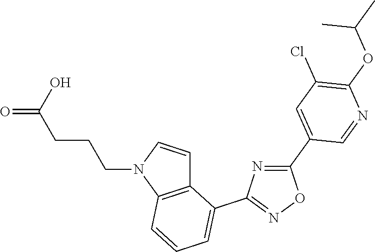 [SMILES: CC(C)Oc1ncc(-c2nc(-c3cccc4c3ccn4CCCC(=O)O)no2)cc1Cl] |
| 20 | CS-0777 | Agonist | 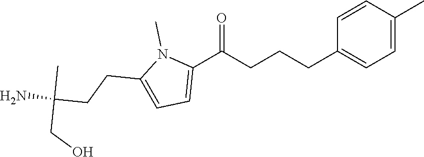 [SMILES: Cc1ccc(CCCC(=O)c2ccc(CC[C@@](C)(N)CO)n2C)cc1] |
| 21 | VPC22277 | Agonist | 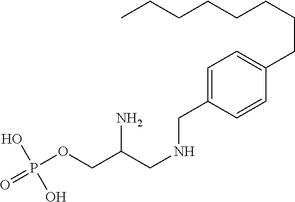 [SMILES: CCCCCCCCc1ccc(CNCC(N)COP(=O)(O)O)cc1] |
| 22 | Carbamoyl- nicotinamide | Agonist | 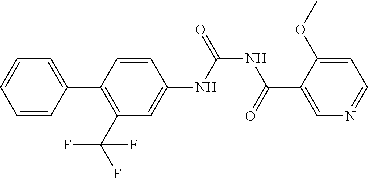 [SMILES: COc1ccncc1C(=O)NC(=O)Nc1ccc(-c2ccccc2)c(C(F)(F)F)c1] |
| 23 | I-FTY720- vinylphospho- nate | Agonist | 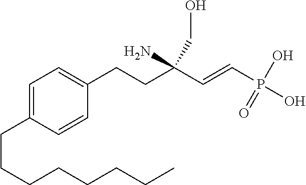 [SMILES: CCCCCCCCc1ccc(CC[C@@](N)(/C=C/P(=O)(O)O)CO)cc1] |
| 24 | (S)-FTY720- phosphate | Agonist | 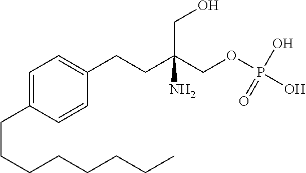 [SMILES: CCCCCCCCc1ccc(CC[C@](N)(CO)COP(=O)(O)O)cc1] |
| 25 | (R)-FTY720- phosphonate | Agonist | 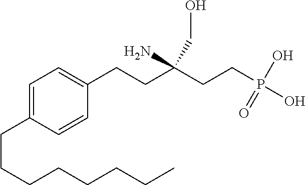 [SMILES: CCCCCCCCc1ccc(CC[C@](N)(CO)CCP(=O)(O)O)cc1] |
| 26 | BMS-986104 | Agonist | 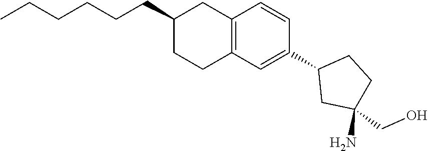 [SMILES: CCCCCC[C@@H]1CCc2cc([C@@H]3CC[C@](N)(CO)C3)ccc2C1] |
| 27 | BMS-824 | Agonist | 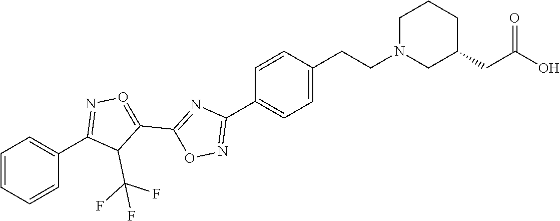 |
| 28 | | Agonist | 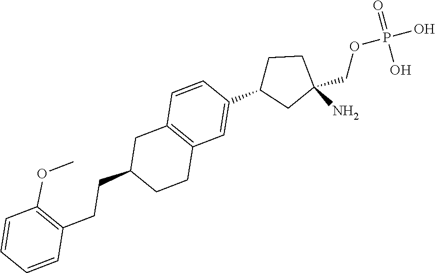 [SMILES: COc1ccccc1CC[C@@H]1CCc2cc([C@@H]3CC[C@](N)(COP(=O)(O)O)C3)ccc2C1] |
| 29 | | Agonist | 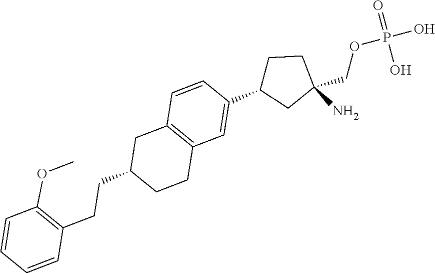 [SMILES: COc1ccccc1CC[C@H]1CCc2cc([C@@H]3CC[C@](N)(COP(=O)(O)O)C3)ccc2C1] |
| 30 | | Agonist | 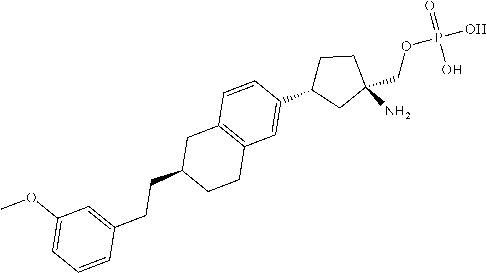 [SMILES: COc1cccc(CC[C@@H]2CCc3cc([C@@H]4CC[C@](N)(COP(=O)(O)O)C4)ccc3C2)c1] |
| 31 | | Agonist | 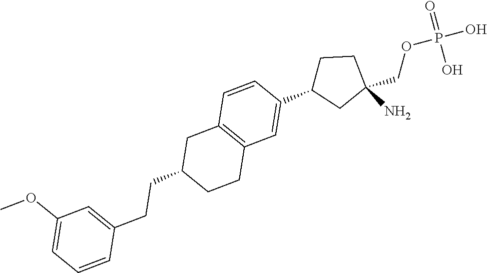 [SMILES: COc1cccc(CC[C@H]2CCc3cc([C@@H]4CC[C@](N)(COP(=O)(O)O)C4)ccc3C2)c1] |
| 32 | | Agonist | 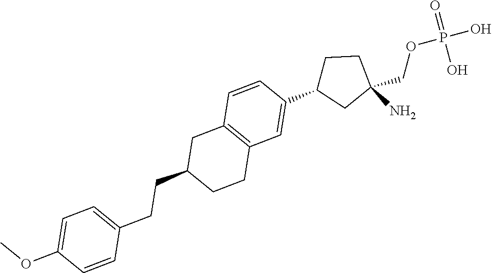 [SMILES: COc1ccc(CC[C@@H]2CCc3cc([C@@H]4CC[C@](N)(COP(=O)(O)O)C4)ccc3C2)cc1] |
| 33 | | Agonist | 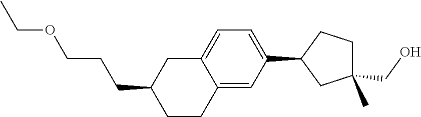 [SMILES: CCOCCC[C@@H]1CCc2cc([C@H]3CC[C@](C)(CO)C3)ccc2C1] |
| 34 | | Agonist | 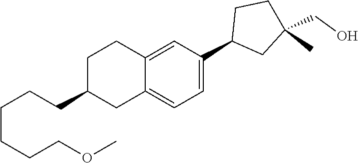 [SMILES: COCCCCCC[C@H]1CCc2cc([C@H]3CC[C@](C)(CO)C3)ccc2C1] |
| 35 | 1-P (BMS- 986104- Phosphate) | Agonist | 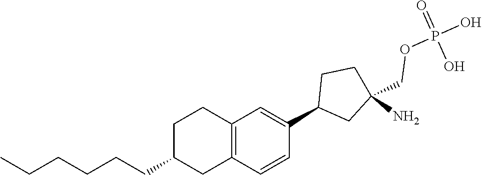 [SMILES: CCCCCC[C@@H]1CCc2cc([C@H]3CC[C@](N)(COP(=O)(O)O)C3)ccc2C1] |
| 36 | BMS-986166 | Agonist | 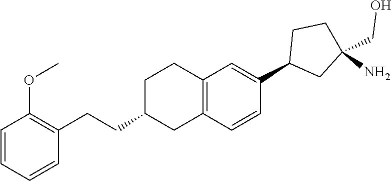 [SMILES: COc1ccccc1CC[C@@H]1CCc2cc([C@H]3CC[C@](N)(CO)C3)ccc2C1] |
| 37 | | Agonist | 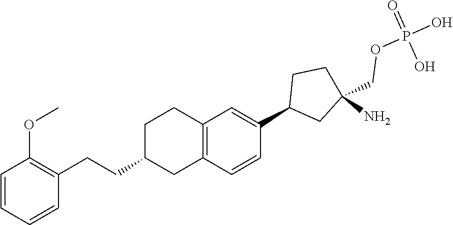 [SMILES: COc1ccccc1CC[C@@H]1CCc2cc([C@H]3CC[C@](N)(COP(=O)(O)O)C3)ccc2C1] |
| 38 | | Agonist | 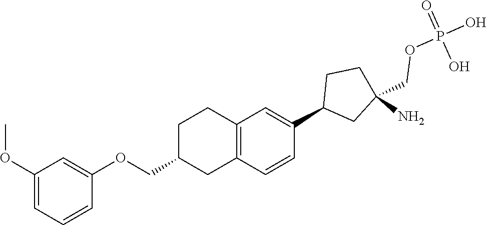 [SMILES: COc1cccc(OC[C@@H]2CCc3cc([C@H]4CC[C@](N)(COP(=O)(O)O)C4)ccc3C2)c1] |
| 39 | | Agonist | 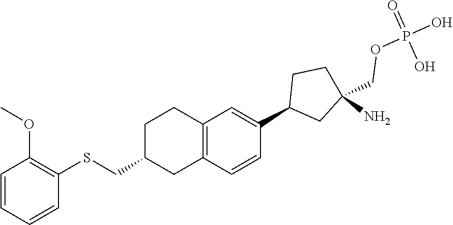 [SMILES: COc1ccccc1SC[C@@H]1CCc2cc([C@H]3CC[C@](N)(COP(=O)(O)O)C3)ccc2C1] |
| 40 | | Agonist | 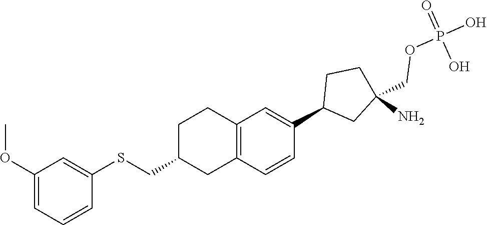 [SMILES: COc1cccc(SC[C@@H]2CCc3cc([C@H]4CC[C@](N)(COP(=O)(O)O)C4)ccc3C2)c1] |
| 41 | | Agonist | 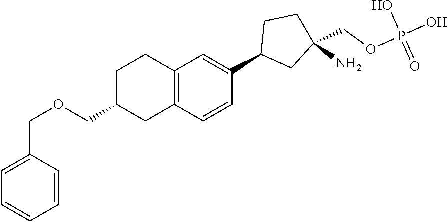 [SMILES: N[C@]1(COP(=O)(O)O)CC[C@H](c2ccc3c(c2)CC[C@@H](COCc2ccccc2)C3)C1] |
| 42 | CAY10444 | Antagonist | 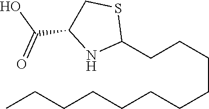 [SMILES: CCCCCCCCCCCC1N[C@H](C(=O)O)CS1] |
| 43 | etrasimod | Antagonist | 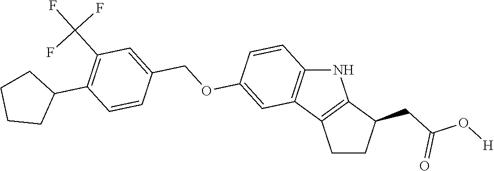 [SMILES: O=C(O)C[C@H]1CCc2c1[nH]c1ccc(OCc3ccc(C4CCCC4)c(C(F)(F)F)c3)cc21] |
| 44 | Amiselimod (MT-1303) | Antagonist | 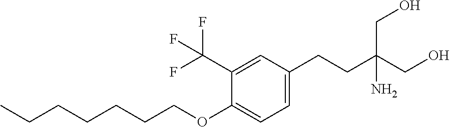 [SMILES: CCCCCCCOc1ccc(CCC(N)(CO)CO)cc1C(F)(F)F] |
| 45 | W146 | Antagonist | 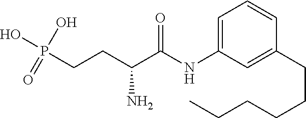 [SMILES: CCCCCCc1cccc(NC(=O)[C@H](N)CCP(=O)(O)O)c1] |
| 46 | VPC 23019 | Antagonist | 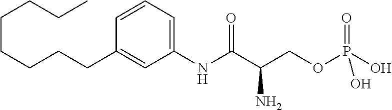 [SMILES: CCCCCCCCc1cccc(NC(=O)[C@H](N)COP(=O)(O)O)c1] |
| 47 | JTE 013 | Antagonist | 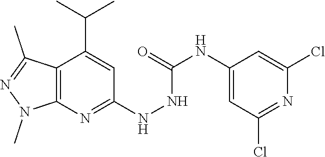 [SMILES: Cc1nn(C)c2nc(NNC(=O)Nc3cc(Cl)nc(Cl)c3)cc(C(C)C)c12] |
| 48 | AB1 | Antagonist | 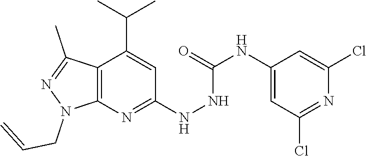 [SMILES: C=CCn1nc(C)c2c(C(C)C)cc(NNC(=O)Nc3cc(Cl)nc(Cl)c3)nc21] |
| 49 | CYM 50260 | Antagonist | 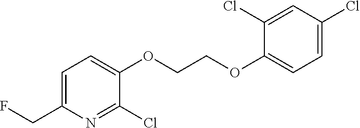 [SMILES: FCc1ccc(OCCOc2ccc(Cl)cc2Cl)c(Cl)n1] |
| 50 | CYM 50308 | Antagonist | 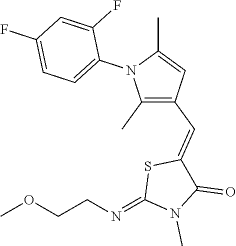 [SMILES: COCC/N=C1\S/C(=C\c2cc(C)n(-c3ccc(F)cc3F)c2C)C(=O)N1C] |
| 51 | AAL-R | Antagonist | 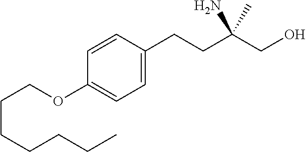 [SMILES: CCCCCCCOc1ccc(CC[C@@](C)(N)CO)cc1] |
| 52 | VPC03090 | Antagonist | 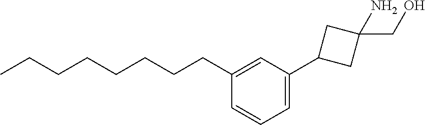 [SMILES: CCCCCCCCc1cccc(C2CC(N)(CO)C2)c1] |
| 53 | VPC44116 | Antagonist | 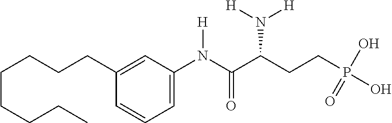 [SMILES: CCCCCCCCc1cccc(NC(=O)[C@H](N)CCP(=O)(O)O)c1] |
| 54 | NIBR-0213 | Antagonist | 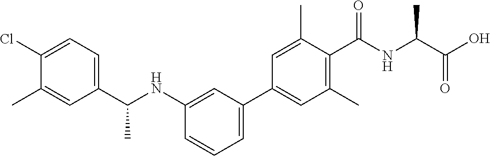 [SMILES: Cc1cc([C@@H](C)Nc2cccc(-c3cc(C)c(C(=O)N[C@@H](C)C(=O)O)c(C)c3)c2)ccc1Cl] |
| 55 | (S)-FTY720- Vinylphospho- nate | Antagonist | 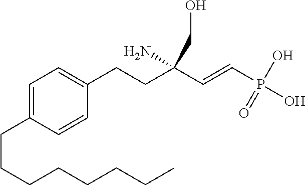 [SMILES: CCCCCCCCc1ccc(CC[C@](N)(/C=C/P(=O)(O)O)CO)cc1] |
| 56 | VPC22173 | | 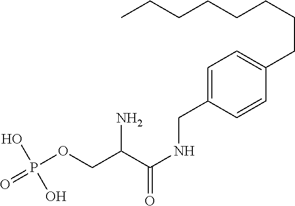 [SMILES: CCCCCCCCc1ccc(CNC(=O)C(N)COP(=O)(O)O)cc1] |
| 57 | | | 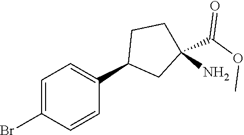 [SMILES: COC(=O)[C@@]1(N)CC[C@H](c2ccc(Br)cc2)C1] |
| 58 | | | 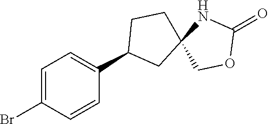 [SMILES: O=C1N[C@@]2(CC[C@H](c3ccc(Br)cc3)C2)CO1] |
| 59 | | | 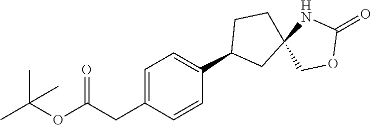 [SMILES: CC(C)(C)OC(=O)Cc1ccc([C@H]2CC[C@]3(COC(=O)N3)C2)cc1] |
| 60 | | | 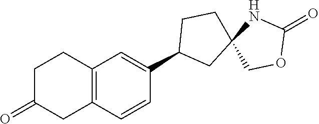 [SMILES: O=C1CCc2cc([C@H]3CC[C@]4(COC(=O)N4)C3)ccc2C1] |
| 61 | | | 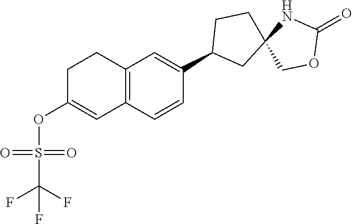 [SMILES: O=C1N[C@@]2(CC[C@H](c3ccc4c(c3)CCC(OS(=O)(=O)C(F)(F)F)=C4)C2)CO1] |
| 62 | | | 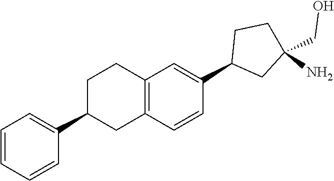 [SMILES: N[C@]1(CO)CC[C@H](c2ccc3c(c2)CC[C@H](c2ccccc2)C3)C1] |
| 63 | | | 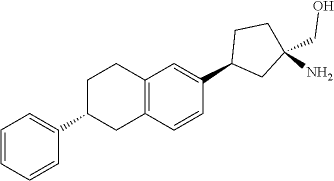 [SMILES: N[C@]1(CO)CC[C@H](c2ccc3c(c2)CC[C@@H](c2ccccc2)C3)C1] |
| 64 | | | 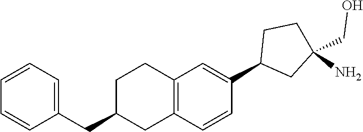 [SMILES: N[C@]1(CO)CC[C@H](c2ccc3c(c2)CC[C@H](Cc2ccccc2)C3)C1] |
| 65 | | | 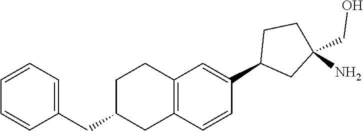 [SMILES: N[C@]1(CO)CC[C@H](c2ccc3c(c2)CC[C@@H](Cc2ccccc2)C3)C1] |
| 66 | | | 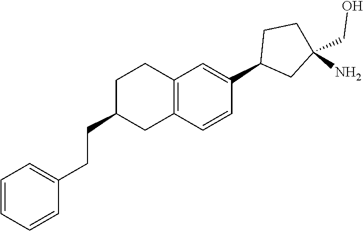 [SMILES: N[C@]1(CO)CC[C@H](c2ccc3c(c2)CC[C@H](CCc2ccccc2)C3)C1] |
| 67 | | | 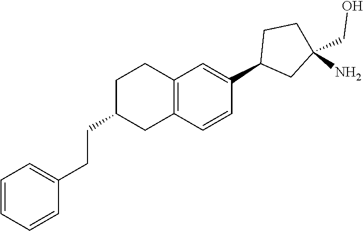 [SMILES: N[C@]1(CO)CC[C@H](c2ccc3c(c2)CC[C@@H](CCc2ccccc2)C3)C1] |
| 68 | | | 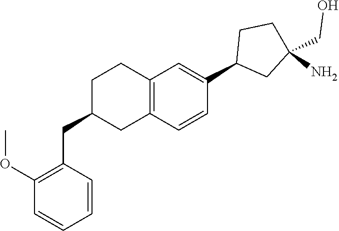 [SMILES: COc1ccccc1C[C@H]1CCc2cc([C@H]3CC[C@](N)(CO)C3)ccc2C1] |
| 69 | | | 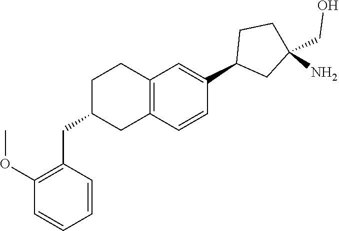 [SMILES: COc1ccccc1C[C@@H]1CCc2cc([C@H]3CC[C@](N)(CO)C3)ccc2C1] |
| 70 | | | 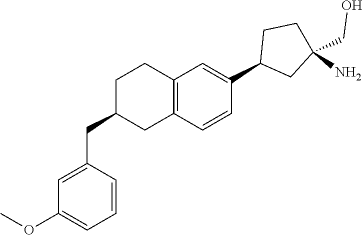 [SMILES: COc1cccc(C[C@H]2CCc3cc([C@H]4CC[C@](N)(CO)C4)ccc3C2)c1] |
| 71 | | | 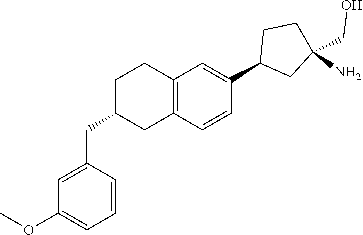 [SMILES: COc1cccc(C[C@@H]2CCc3cc([C@H]4CC[C@](N)(CO)C4)ccc3C2)c1] |
| 72 | | | 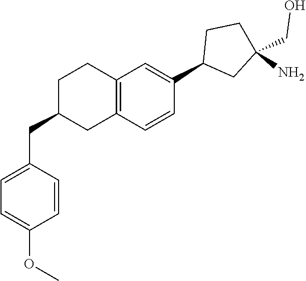 [SMILES: COc1ccc(C[C@H]2CCc3cc([C@H]4CC[C@](N)(CO)C4)ccc3C2)cc1] |
| 73 | | | 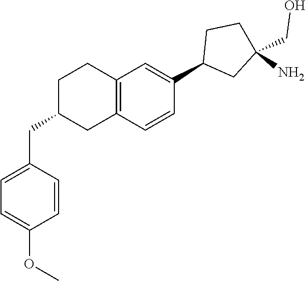 [SMILES: COc1ccc(C[C@@H]2CCc3cc([C@H]4CC[C@](N)(CO)C4)ccc3C2)cc1] |
| 74 | | | 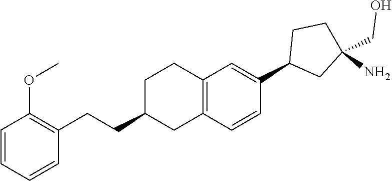 [SMILES: COc1ccccc1CC[C@H]1CCc2cc([C@H]3CC[C@](N)(CO)C3)ccc2C1] |
| 75 | | | 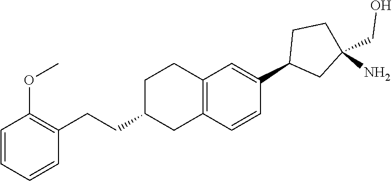 [SMILES: COc1ccccc1CC[C@@H]1CCc2cc([C@H]3CC[C@](N)(CO)C3)ccc2C1] |
| 76 | | | 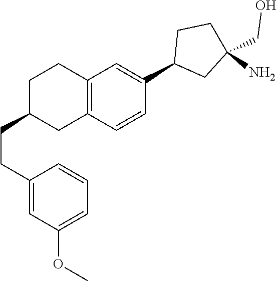 [SMILES: COc1cccc(CC[C@H]2CCc3cc([C@H]4CC[C@](N)(CO)C4)ccc3C2)c1] |
| 77 | | | 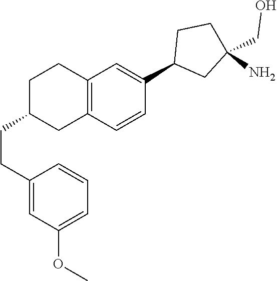 [SMILES: COc1cccc(CC[C@@H]2CCc3cc([C@H]4CC[C@](N)(CO)C4)ccc3C2)c1] |
| 78 | | | 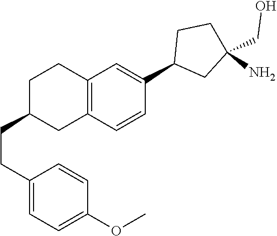 [SMILES: COc1ccc(CC[C@H]2CCc3cc([C@H]4CC[C@](N)(CO)C4)ccc3C2)cc1] |
| 79 | | | 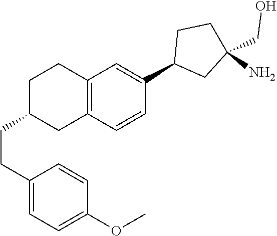 [SMILES: COc1ccc(CC[C@@H]2CCc3cc([C@H]4CC[C@](N)(CO)C4)ccc3C2)cc1] |
| 80 | | | 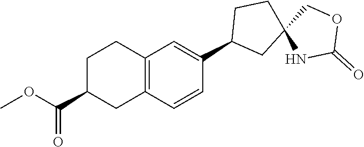 [SMILES: COC(=O)[C@H]1CCc2cc([C@H]3CC[C@]4(COC(=O)N4)C3)ccc2C1] |
| 81 | | | 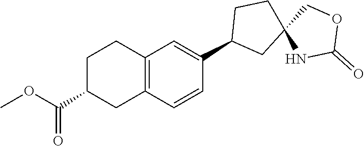 [SMILES: COC(=O)[C@@H]1CCc2cc([C@H]3CC[C@]4(COC(=O)N4)C3)ccc2C1] |
| 82 | | | 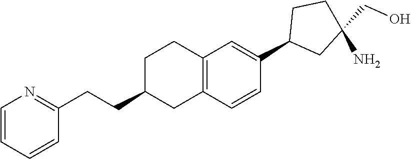 [SMILES: N[C@]1(CO)CC[C@H](c2ccc3c(c2)CC[C@H](CCc2ccccn2)C3)C1] |
| 83 | | | 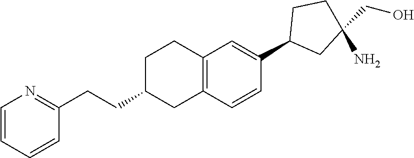 [SMILES: N[C@]1(CO)CC[C@H](c2ccc3c(c2)CC[C@@H](CCc2ccccn2)C3)C1] |
| 84 | | | 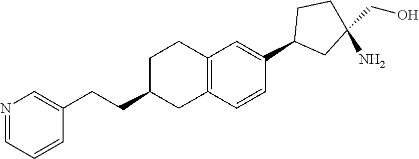 [SMILES: N[C@]1(CO)CC[C@H](c2ccc3c(c2)CC[C@H](CCc2cccnc2)C3)C1] |
| 85 | | | 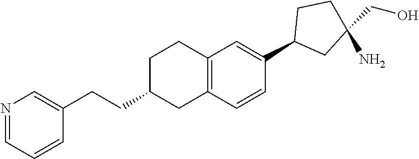 [SMILES: N[C@]1(CO)CC[C@H](c2ccc3c(c2)CC[C@@H](CCc2cccnc2)C3)C1] |
| 86 | | | 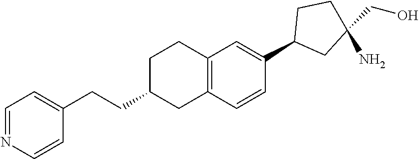 [SMILES: N[C@]1(CO)CC[C@H](c2ccc3c(c2)CC[C@@H](CCc2ccncc2)C3)C1] |
| 87 | | | 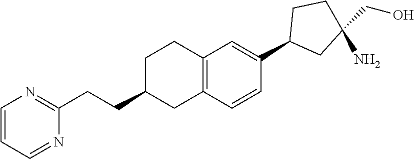 [SMILES: N[C@]1(CO)CC[C@H](c2ccc3c(c2)CC[C@H](CCc2ncccn2)C3)C1] |
| 88 | | | 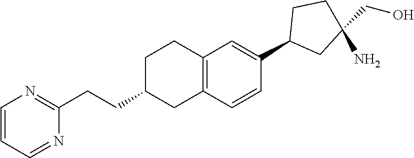 [SMILES: N[C@]1(CO)CC[C@H](c2ccc3c(c2)CC[C@@H](CCc2ncccn2)C3)C1] |
| 89 | | | 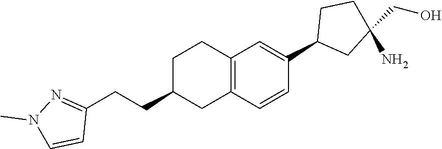 [SMILES: Cn1ccc(CC[C@H]2CCc3cc([C@H]4CC[C@](N)(CO)C4)ccc3C2)n1] |
| 90 | | | 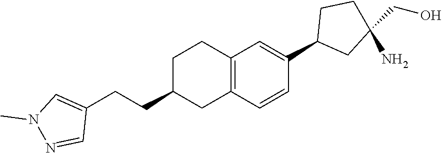 [SMILES: Cn1cc(CC[C@H]2CCc3cc([C@H]4CC[C@](N)(CO)C4)ccc3C2)cn1] |
| 91 | | | 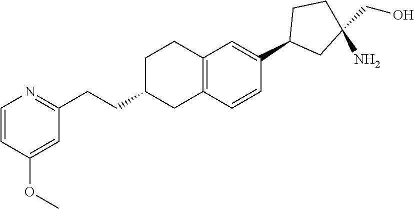 [SMILES: COc1ccnc(CC[C@@H]2CCc3cc([C@H]4CC[C@](N)(CO)C4)ccc3C2)c1] |
| 92 | | | 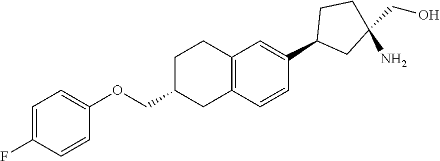 [SMILES: N[C@]1(CO)CC[C@H](c2ccc3c(c2)CC[C@@H](COc2ccc(F)cc2)C3)C1] |
| 93 | | | 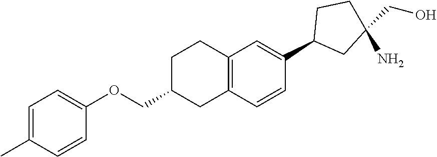 [SMILES: Cc1ccc(OC[C@@H]2CCc3cc([C@H]4CC[C@](N)(CO)C4)ccc3C2)cc1] |
| 94 | | | 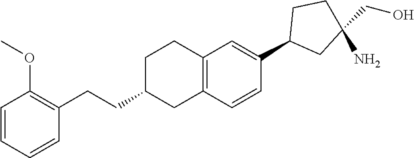 [SMILES: COc1ccccc1CC[C@@H]1CCc2cc([C@H]3CC[C@](N)(CO)C3)ccc2C1] |
| 95 | | | 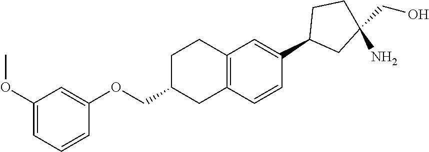 [SMILES: COc1cccc(OC[C@@H]2CCc3cc([C@H]4CC[C@](N)(CO)C4)ccc3C2)c1] |
| 96 | | | 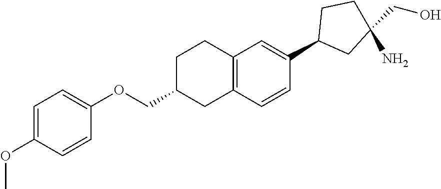 [SMILES: COc1ccc(OC[C@@H]2CCc3cc([C@H]4CC[C@](N)(CO)C4)ccc3C2)cc1] |
| 97 | | | 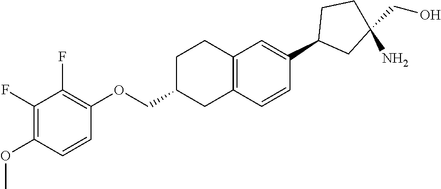 [SMILES: COc1ccc(OC[C@@H]2CCc3cc([C@H]4CC[C@](N)(CO)C4)ccc3C2)c(F)c1F] |
| 98 | | | 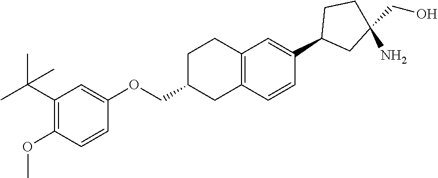 [SMILES: COc1ccc(OC[C@@H]2CCc3cc([C@H]4CC[C@](N)(CO)C4)ccc3C2)cc1C(C)(C)C] |
| 99 | | | 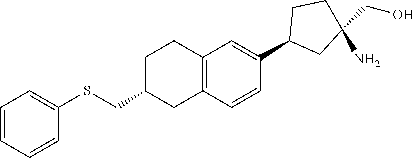 [SMILES: N[C@]1(CO)CC[C@H](c2ccc3c(c2)CC[C@@H](CSc2ccccc2)C3)C1] |
| 100 | | | 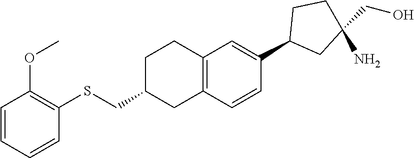 [SMILES: COc1ccccc1SC[C@@H]1CCc2cc([C@H]3CC[C@](N)(CO)C3)ccc2C1] |
| 101 | | | 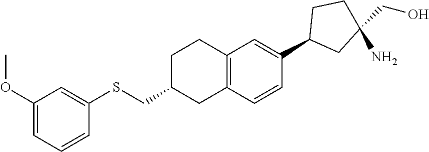 [SMILES: COc1cccc(SC[C@@H]2CCc3cc([C@H]4CC[C@](N)(CO)C4)ccc3C2)c1] |
| 102 | | | 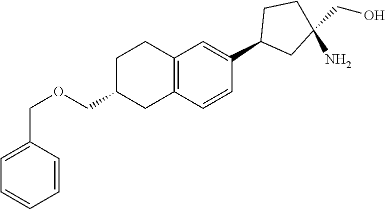 [SMILES: N[C@]1(CO)CC[C@H](c2ccc3c(c2)CC[C@@H](COCc2ccccc2)C3)C1] |
| 103 | | | 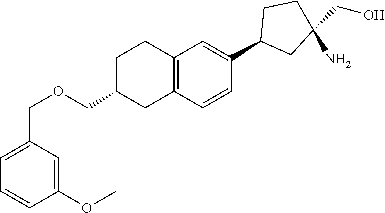 [SMILES: COc1cccc(COC[C@@H]2CCc3cc([C@H]4CC[C@](N)(CO)C4)ccc3C2)c1] |
| 104 | | | 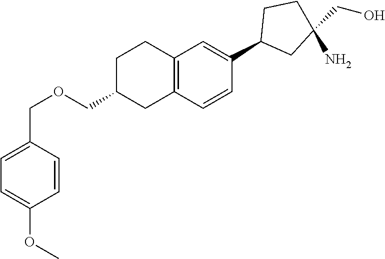 [SMILES: COc1ccc(COC[C@@H]2CCc3cc([C@H]4CC[C@](N)(CO)C4)ccc3C2)cc1] |
| 105 | | | 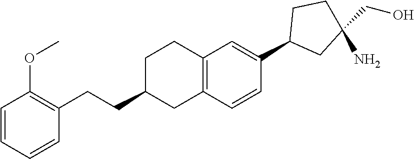 [SMILES: COc1ccccc1CC[C@H]1CCc2cc([C@H]3CC[C@](N)(CO)C3)ccc2C1] |
| 106 | | | 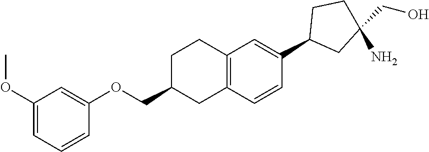 [SMILES: COc1cccc(OC[C@H]2CCc3cc([C@H]4CC[C@](N)(CO)C4)ccc3C2)c1] |
| 107 | | | 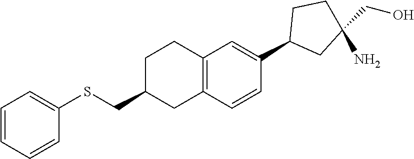 [SMILES: N[C@]1(CO)CC[C@H](c2ccc3c(c2)CC[C@H](CSc2ccccc2)C3)C1] |
| 108 | | | 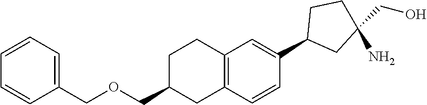 [SMILES: N[C@]1(CO)CC[C@H](c2ccc3c(c2)CC[C@H](COCc2ccccc2)C3)C1] |
| 109 | | | 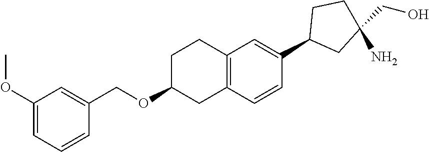 [SMILES: COc1cccc(CO[C@H]2CCc3cc([C@H]4CC[C@](N)(CO)C4)ccc3C2)c1] |
| 110 | | | 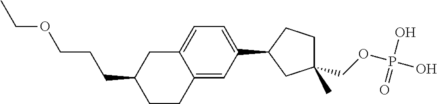 [SMILES: CCOCCC[C@@H]1CCc2cc([C@H]3CC[C@](C)(COP(=O)(O)O)C3)ccc2C1] |
| 111 | | | 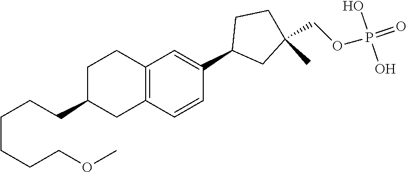 [SMILES: COCCCCCC[C@H]1CCc2cc([C@H]3CC[C@](C)(COP(=O)(O)O)C3)ccc2C1] |
| 112 | | | 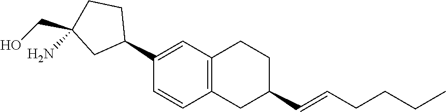 [SMILES: CCCC/C=C/[C@@H]1CCc2cc([C@@H]3CC[C@](N)(CO)C3)ccc2C1] |
| 113 | | | 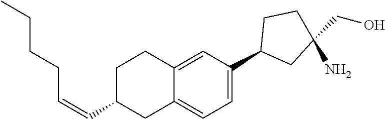 [SMILES: CCCC/C=C\[C@@H]1CCc2cc([C@H]3CC[C@](N)(CO)C3)ccc2C1] |
| 114 | | | 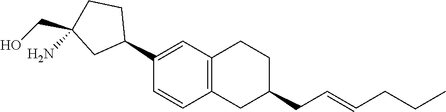 [SMILES: CCC/C=C/C[C@@H]1CCc2cc([C@@H]3CC[C@](N)(CO)C3)ccc2C1] |
| 115 | | | 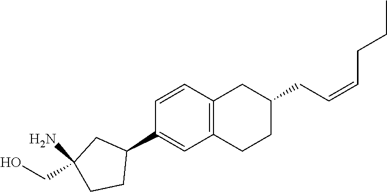 [SMILES: CCC/C=C\C[C@@H]1CCc2cc([C@H]3CC[C@](N)(CO)C3)ccc2C1] |
| 116 | | | 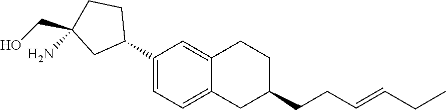 [SMILES: CC/C=C/CC[C@@H]1CCc2cc([C@H]3CC[C@](N)(CO)C3)ccc2C1] |
| 117 | | | 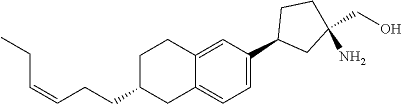 [SMILES: CC/C=C\CC[C@@H]1CCc2cc([C@H]3CC[C@](N)(CO)C3)ccc2C1] |
| 118 | | | 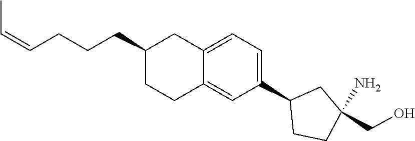 [SMILES: C/C=C\CCC[C@@H]1CCc2cc([C@@H]3CC[C@](N)(CO)C3)ccc2C1] |
| 119 | | | 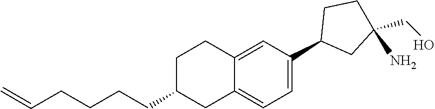 [SMILES: C=CCCCC[C@@H]1CCc2cc([C@H]3CC[C@](N)(CO)C3)ccc2C1] |
| 120 | | | 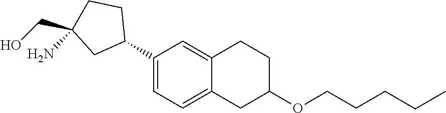 [SMILES: CCCCCOC1CCc2cc([C@H]3CC[C@](N)(CO)C3)ccc2C1] |
| 121 | | | 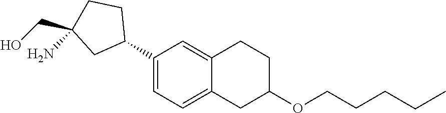 [SMILES: CCCCCOC1CCc2cc([C@H]3CC[C@](N)(CO)C3)ccc2C1] |
| 122 | | | 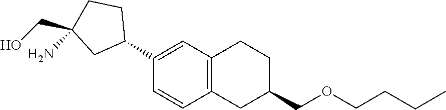 [SMILES: CCCCOC[C@@H]1CCc2cc([C@H]3CC[C@](N)(CO)C3)ccc2C1] |
| 123 | | | 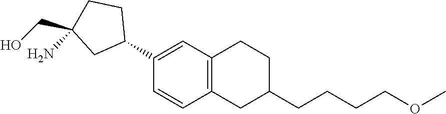 [SMILES: COCCCCC1CCc2cc([C@H]3CC[C@](N)(CO)C3)ccc2C1] |
| 124 | | | 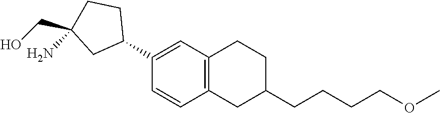 [SMILES: COCCCCC1CCc2cc([C@H]3CC[C@](N)(CO)C3)ccc2C1] |
| 125 | | | 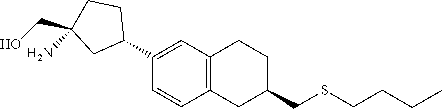 [SMILES: CCCCSC[C@@H]1CCc2cc([C@H]3CC[C@](N)(CO)C3)ccc2C1] |
| 126 | | | 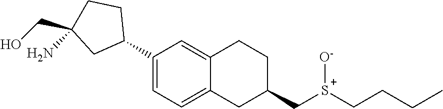 [SMILES: CCCC[S+]([O-])C[C@@H]1CCc2cc([C@H]3CC[C@](N)(CO)C3)ccc2C1] |
| 127 | | | 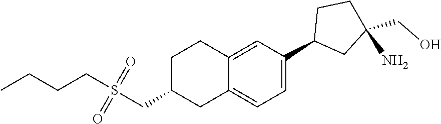 [SMILES: CCCCS(=O)(=O)C[C@@H]1CCc2cc([C@H]3CC[C@](N)(CO)C3)ccc2C1] |
| 128 | | | 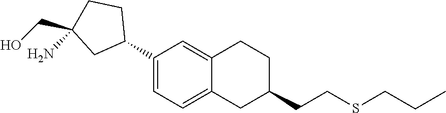 [SMILES: CCCSCC[C@@H]1CCc2cc([C@H]3CC[C@](N)(CO)C3)ccc2C1] |
| 129 | | | 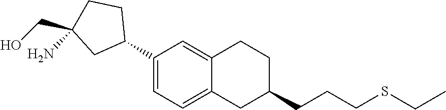 [SMILES: CCSCCC[C@@H]1CCc2cc([C@H]3CC[C@](N)(CO)C3)ccc2C1] |
| 130 | | | 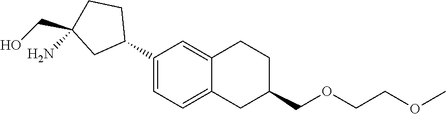 [SMILES: COCCOC[C@@H]1CCc2cc([C@H]3CC[C@](N)(CO)C3)ccc2C1] |
| 131 | | | 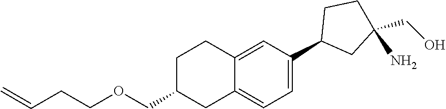 [SMILES: C=CCCOC[C@@H]1CCc2cc([C@H]3CC[C@](N)(CO)C3)ccc2C1] |
| 132 | | | 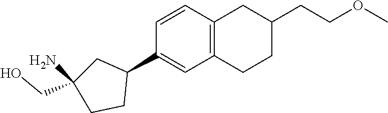 [SMILES: COCCC1CCc2cc([C@H]3CC[C@](N)(CO)C3)ccc2C1] |
| 133 | | | 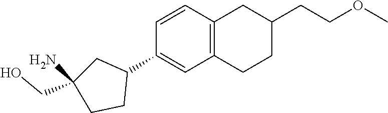 |
| 134 | | | 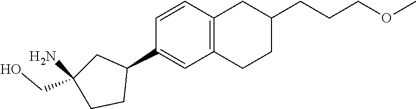 [SMILES: COCCCC1CCc2cc([C@H]3CC[C@](N)(CO)C3)ccc2C1] |
| 135 | | | 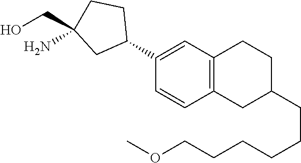 [SMILES: COCCCCCCC1CCc2cc([C@H]3CC[C@](N)(CO)C3)ccc2C1] |
| 136 | | | 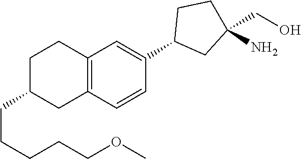 [SMILES: COCCCCC[C@@H]1CCc2cc([C@@H]3CC[C@](N)(CO)C3)ccc2C1] |
| 137 | | | 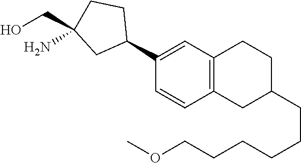 [SMILES: COCCCCCCC1CCc2cc([C@@H]3CC[C@](N)(CO)C3)ccc2C1] |
| 138 | | | 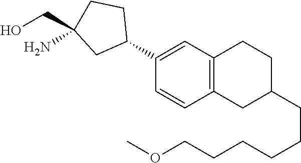 [SMILES: COCCCCCCC1CCc2cc([C@H]3CC[C@](N)(CO)C3)ccc2C1] |
| 139 | | | 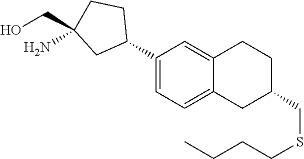 [SMILES: CCCCSC[C@H]1CCc2cc([C@H]3CC[C@](N)(CO)C3)ccc2C1] |
| 140 | | | 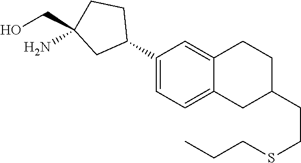 [SMILES: CCCSCCC1CCc2cc([C@H]3CC[C@](N)(CO)C3)ccc2C1] |
| 141 | | | 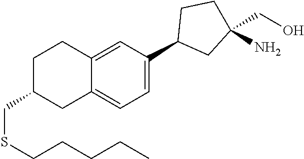 [SMILES: CCCCCSC[C@@H]1CCc2cc([C@H]3CC[C@](N)(CO)C3)ccc2C1] |
| 142 | | | 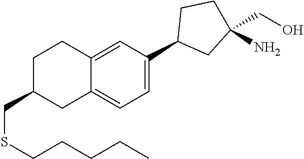 [SMILES: CCCCCSC[C@H]1CCc2cc([C@H]3CC[C@](N)(CO)C3)ccc2C1] |
| 143 | | | 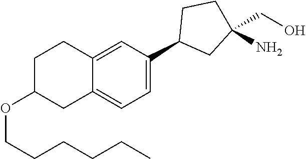 [SMILES: CCCCCCOC1CCc2cc([C@H]3CC[C@](N)(CO)C3)ccc2C1] |
| 144 | | | 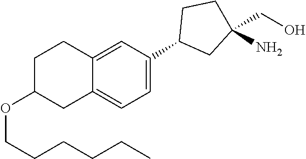 [SMILES: CCCCCCOC1CCc2cc([C@@H]3CC[C@](N)(CO)C3)ccc2C1] |
| 145 | | | 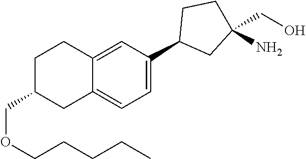 [SMILES: CCCCCOC[C@@H]1CCc2cc([C@H]3CC[C@](N)(CO)C3)ccc2C1] |
| 146 | | | 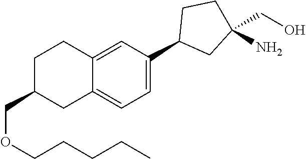 [SMILES: CCCCCOC[C@H]1CCc2cc([C@H]3CC[C@](N)(CO)C3)ccc2C1] |
| 147 | | | 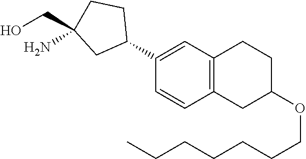 [SMILES: CCCCCCCOC1CCc2cc([C@H]3CC[C@](N)(CO)C3)ccc2C1] |
| 148 | | | 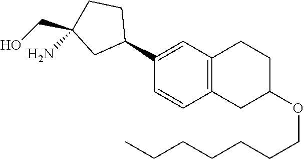 [SMILES: CCCCCCCOC1CCc2cc([C@@H]3CC[C@](N)(CO)C3)ccc2C1] |
| 149 | | | 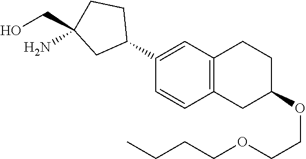 [SMILES: CCCCOCCO[C@@H]1CCc2cc([C@H]3CC[C@](N)(CO)C3)ccc2C1] |
| 150 | | | 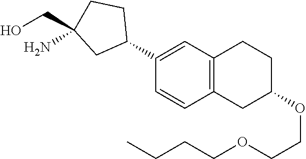 [SMILES: CCCCOCCO[C@H]1CCc2cc([C@H]3CC[C@](N)(CO)C3)ccc2C1] |
| 151 | | | 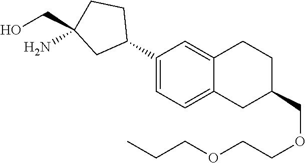 [SMILES: CCCOCCOC[C@@H]1CCc2cc([C@H]3CC[C@](N)(CO)C3)ccc2C1] |
| 152 | | | 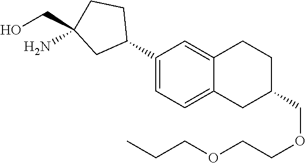 [SMILES: CCCOCCOC[C@H]1CCc2cc([C@H]3CC[C@](N)(CO)C3)ccc2C1] |
| 153 | CYM-5441 | Agonist | 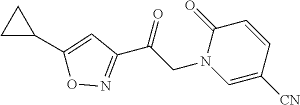 [SMILES: N#Cc1ccc(=O)n(CC(=O)c2cc(C3CC3)on2)c1] |
| 154 | CYM 5520 | Agonist | 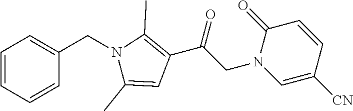 [SMILES: Cc1cc(C(=O)Cn2cc(C#N)ccc2=O)c(C)n1Cc1ccccc1] |
| 155 | A971432 | Agonist | 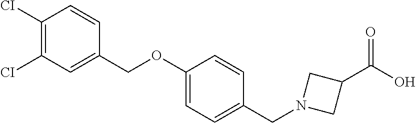 |
| 156 | AKP-11 | Agonist | 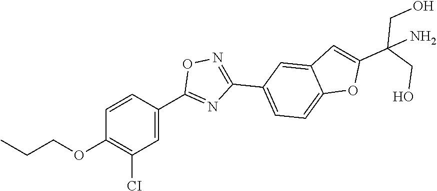 |
| 157 | Cenerimod (ACT-334441) | Agonist | 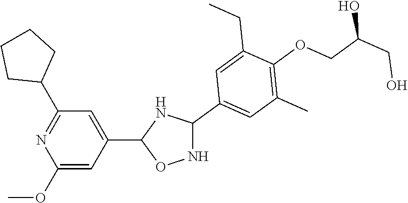 [SMILES: CCc1cc(C2NOC(c3cc(OC)nc(C4CCCC4)c3)N2)cc(C)c1OC[C@@H](O)CO] |

In some embodiments, the S1PR modulator is as described in U.S. Pat. No. 11,034,691, 10,544,136, 8,796,318 10,323,029, 9,975,863, 8,618,139, 8,399,492, 8,501,726, 8,486,918, 7,220,734, 7,605,171, 7,678,820, 10,323,029, 8,530,503, 8,481,573, 8,466,183, 9,382,217, 7,220,734, 7,605,171, or 9,382,217, or US Pub Nos. US 2015/005703, US 2012/0142745, US 2006/0161005, US 2005/0020837, US 2008/0249093, US 20070043014, US 20080306124, US 2010/0010001, US 2012/0329840, US 2018/0009770, US 2012/0329838, US 2017/0050941, US 2015/0057307, US 2012/0329839, US 2017/0217963, US 2019/0300527, US 2018/0009770, US 2017/0217693, or US 2017/0050941, each of which is hereby incorporated by reference in its entirety.

In some embodiments, the S1PR modulator is an agonist. In some embodiments, the agonist is fingolimod, ozanimod (RPC106340iponimodmod (BAF312), ponesimod (ACT128800), A971432, CYM-5442, S1P, TC-SP 14, SEW 2871, RP 001 hydrochloride, CS 2100, RP 001, ASP4058, AMG 369, AUY954, KRP-203, ceralifimod (ONO-4641), GSK2018682, CS-0777, VPC22277, carbamoyl-nicotinamide, CYM 5441, CYM 5520, A971432, or a pharmaceutically acceptable salt thereof.

In some embodiments, the S1PR modulator is an antagonist. In some embodiments, the antagonist is CAY10444, etrasimod, amiselimod (MT-1303), W146, VPC 23019, JTE 013, AB1, CYM 50260, CYM 50308, AAL-R, VPC03090, VPC44116, NIBR-0213, or a pharmaceutically acceptable salt thereof.

### Formulations and Carriers

This invention describes methods of reducing alcohol intake by administering a pharmaceutical composition containing a S1PR modulator (e.g., a compound of Table 1). The S1PR modulator (e.g., agonist or antagonist) may be formulated with a pharmaceutically acceptable carrier or excipient. A pharmaceutically acceptable carrier or excipient refers to a carrier (e.g., carrier, media, diluent, solvent, vehicle, etc.) which does not significantly interfere with the biological activity or effectiveness of the active ingredient(s) of a pharmaceutical composition and that is not excessively toxic to the host at the concentrations at which it is used or administered. Other pharmaceutically acceptable ingredients may be present in the composition as well. Suitable substances and their use for the formulation of pharmaceutically active compounds are well-known in the art (see, for example, Remington: The Science and Practice of Pharmacy. ²1st Edition. Philadelphia, PA. Lippincott Williams & Wilkins, 2005, for additional discussion of pharmaceutically acceptable substances and methods of preparing pharmaceutical compositions of various types).

### Dosage and Administration

The pharmaceutical compositions used in this invention (e.g., containing a compound of Table 1) can be administered to a subject (e.g., a human subject) in a variety of ways. The compositions must be suitable for the subject receiving the treatment and the mode of administration. Furthermore, the severity of the condition to be treated affects the dosages and routes. The pharmaceutical compositions used in this invention may be administered orally, buccally, sublingually, parenterally, intravenously, subcutaneously, intramedullary, intranasally, as a suppository, using a flash formulation, topically, intradermally, subcutaneously, via pulmonary delivery, via intra-arterial injection, ophthalmically, optically, intrathecally, or via a mucosal route. In some embodiments, the pharmaceutical composition is administered orally.

A dosage regimen may be determined by the clinical indication being addressed, as well as by various patient variables (e.g., weight, age, sex) and clinical presentation (e.g., extent or severity of disease). Furthermore, it is understood that all dosages may be continuously given or divided into dosages given per a given time frame.

### Combination Therapies

As the methods of the invention described herein include reducing alcohol intake, it may be useful to design combination therapies that include two or more pharmaceutical agents (e.g., an SP1R modulator as described herein, e.g., a compound of Table 1, and a second agent). The two or more agents may be administered sequentially, or at substantially the same time. The two or more agents may be administered in the same formulation or as separate formulations.

The combination therapies of the invention may be useful in providing synergistic effects of the two or more pharmaceutical agents. For example, the half maximal inhibitory concentration (IC₅₀) of a certain drug may be lowered upon administration with a second agent. Additionally, the cumulative treatment effect of two drugs in combination may be greater than the sum of the treatment effects of each individual drug. This behavior may be beneficial by lowering the amount of drug required to treat a particular cancer or tumor. When a drug exhibits toxicity, it is preferable to use the lowest dosage possible to achieve a treatment effect to minimize detrimental side effects while still maintaining efficacy. In some instances, the effects of the combination therapy will be synergistic, but the side effects will not.

In some embodiments, an S1PR is administered in combination with a pharmaceutical agent as described in PCT Pub. No. WO2017083795, which is hereby incorporated by reference in its entirety.

### EXAMPLES

The following examples of specific aspects for carrying out the present disclosure are offered for illustrative purposes only and are not intended to limit the scope of the present disclosure in any way.

### Example 1. Sphingosine Signaling is a New Candidate Regulator of Alcohol Sensitivity and Intake

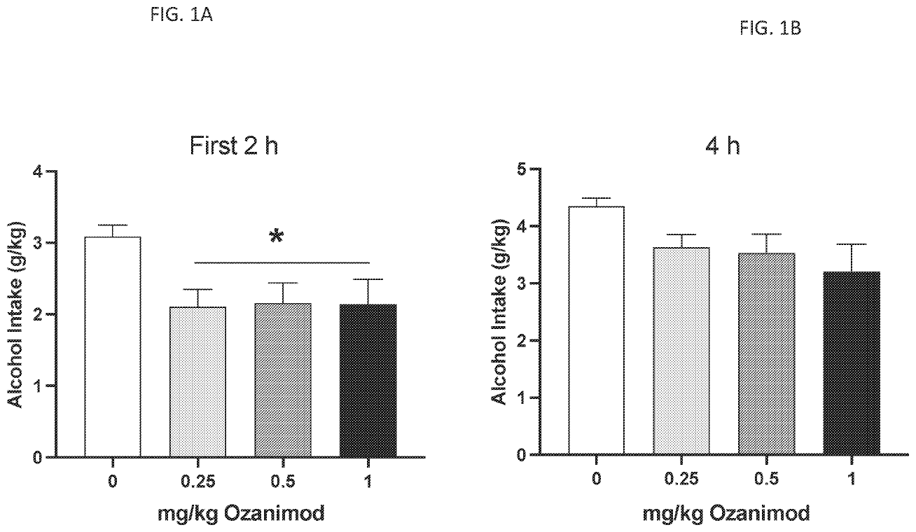

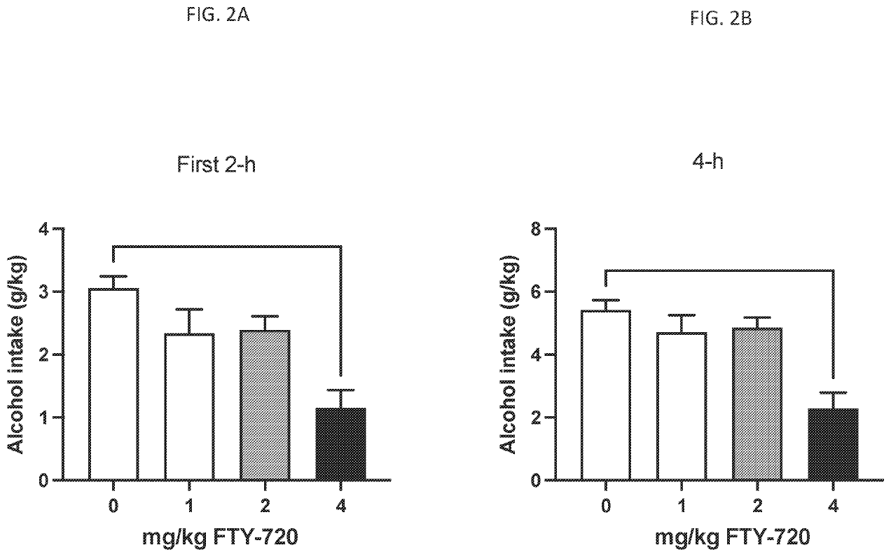

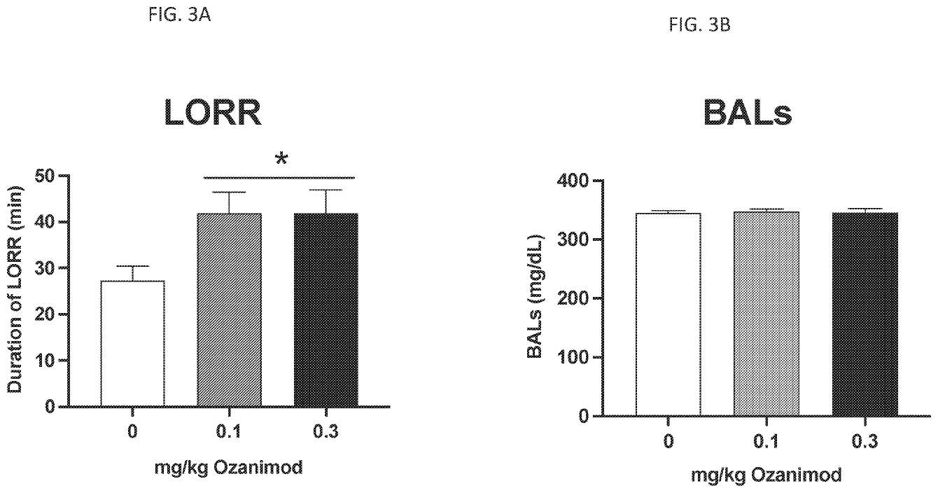

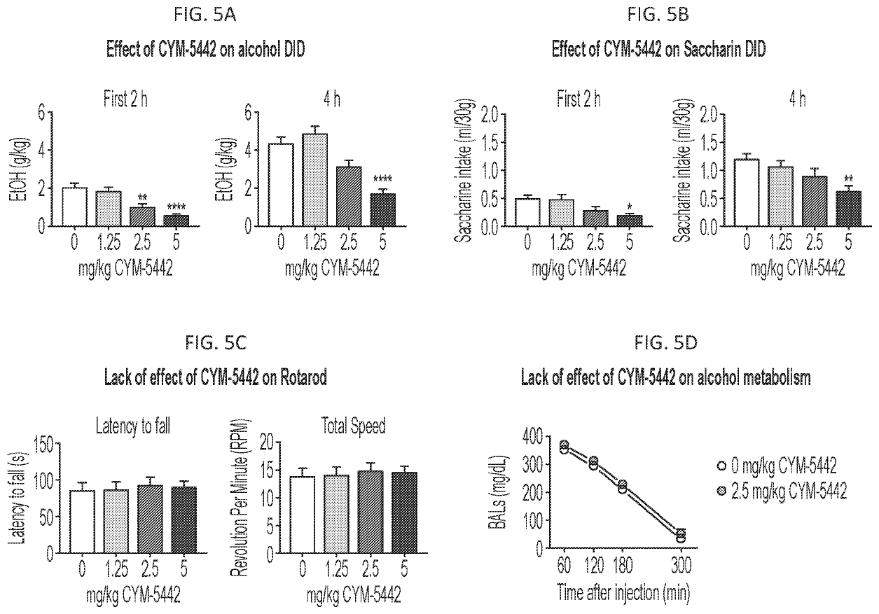

These results indicate that sphingosine-1-phosphate (S1P) signaling is a regulator of alcohol sensitivity and intake. S1P is a lipid mediator that is generated from phosphorylation of sphingosine by the action of sphingosine kinase (SphK) isoenzymes in the brain and other tissues. S1P acts on five G protein-coupled receptors (S1P₁₋₅). Here it is shown that the S1P receptor agonists fingolimod, ozanimod, and CYM-5442 reduced alcohol intake in the drinking in the dark (DID) binge drinking paradigm in C57BL/6J mice (FIGS. 1A, 1B, 2A, 2B, and 5A-5D). The effect of fingolimod and CYM-5442 was more protracted, reflecting its longer circulating half-life. CYM-5442 did not affect motor performance in the Rotarod paradigm, a measure of motor coordination (FIG. 5C) nor alcohol metabolism (FIG. 5D) at a dose of 2.5 mg/kg, which was effective in reducing alcohol.

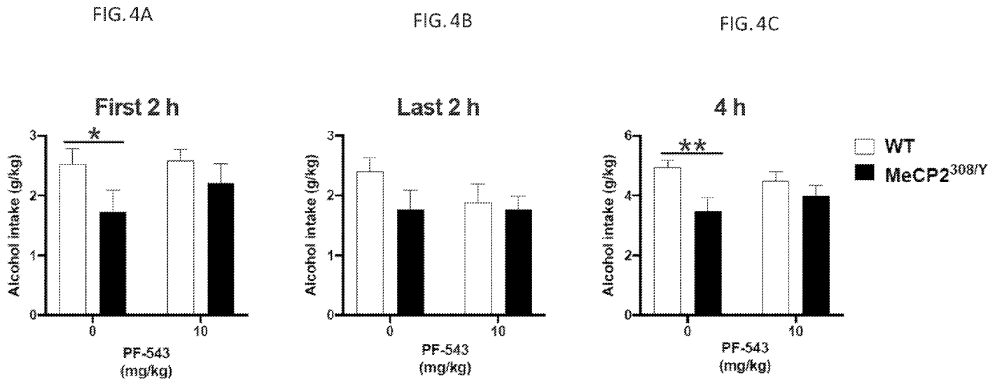

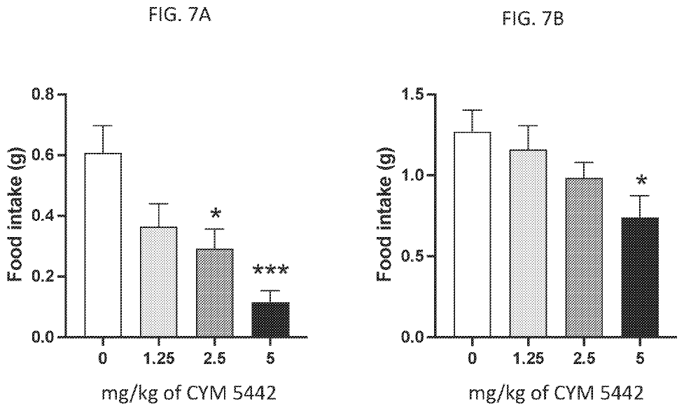

CYM-5442 was also tested on saccharin intake in a design that paralleled DID and showed to reduce saccharin intake, although at a higher dose than was effective in reducing alcohol intake in the DID paradigm (FIG. 5B). Consistently, CYM-5442 also reduced food intake (FIGS. 7A and 7B). Next, the effect of ozanimod was tested on the sensitivity to alcohol's hypnotic action with the loss of righting reflex (LORR) paradigm in C57BL/6J mice. It was observed that ozanimod increased sensitivity to the intoxicating effects of alcohol in the LORR paradigm without altering alcohol metabolism in C57BL/6J mice (FIGS. 3A and 3B). S1P receptor agonists did not produce motor impairment: fingolimod did not impair performance in the rotarod test and did not affect body weight.

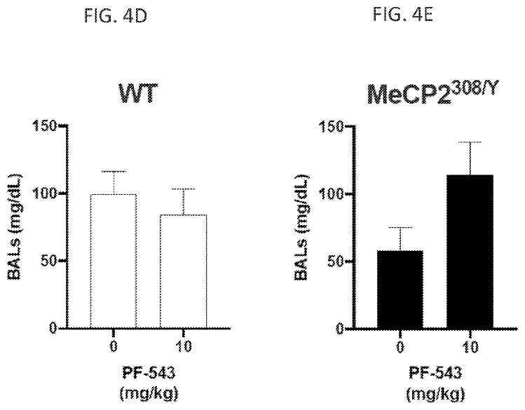

Additionally, fingolimod and ozanimod ameliorated motor deficits in mice with degenerative and inflammatory conditions. It was previously observed that MeCP2^{308/Y} mice, which harbor a truncation of the methyl-CpG binding protein 2 (MeCP2) gene, the causative gene of Rett syndrome, have decreased alcohol intake and increased sensitivity to alcohol. Since increased levels of sphingosine and S1P were previously observed in humans with Rett syndrome, the effect of the SphK inhibitor PF-543 was tested in the DID paradigm in MeCP2^{308/Y} mice. It was observed that PF-543 increased alcohol intake and blood alcohol levels (BAL) in MeCP2^{308/Y} mice in the DID paradigm to the levels of control mice (FIGS. 4A-4E), supporting a role of S1P signaling in alcohol drinking. Altogether these data show that S1P signaling regulates alcohol sensitivity, tolerance, and excessive alcohol intake without affecting alcohol metabolism. Moreover, these data show that modulating S1P signaling can also be used to reduce food intake.

### Example 2. Sphingosine Signaling Reduces Drug Intake

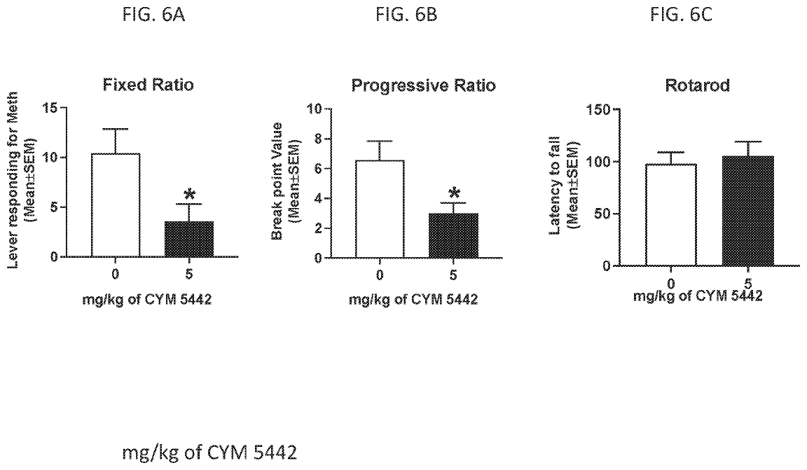

The effect of CYM-5442 was tested on methamphetamine self-administering rats. Male Wistar rats were aseptically implanted with chronic silastic catheters in the right jugular vein and were trained to self-administer methamphetamine (1 h/day at a dose of 0.05 mg/kg/infusion). Rats were trained to self-administer methamphetamine under a fixed-ratio 1 schedule of reinforcement (FR1) whereby one lever press results in the delivery of one methamphetamine infusion (0.05 mg/kg) under 1-h daily sessions. Administration of CYM-5442 (5 mg/Kg) 1 h before the self-administration session significantly reduced voluntary methamphetamine intake under both fixed-ratio 1 schedule of reinforcement (FR1) (FIG. 6A) and under a Progressive Ratio (PR) schedule of reinforcement whereby the response requirement was increased progressively (FIG. 6B). These results indicate that CYM-5442-treated rats had less motivation for methamphetamine as compared to the vehicle-treated rats. At the dose used, CYM-5442 did not affect the rats' motor performance in the Rotarod paradigm (FIG. 6C).

### Example 3. Sphingosine Signaling Reduces Food Intake and Promotes Fat Utilization as Fuel

The acute effect of CYM-5442 on food intake was measured in 4 h sessions. CYM-5442 was administered 1 h before the sessions. CYM-5442 reduced food intake in a dose-dependent manner at the doses of 2.5 and 5 mg/Kg in the first 2 h (FIG. 7A) and at the dose of 5 mg/kg after 4 h (FIG. 7B).

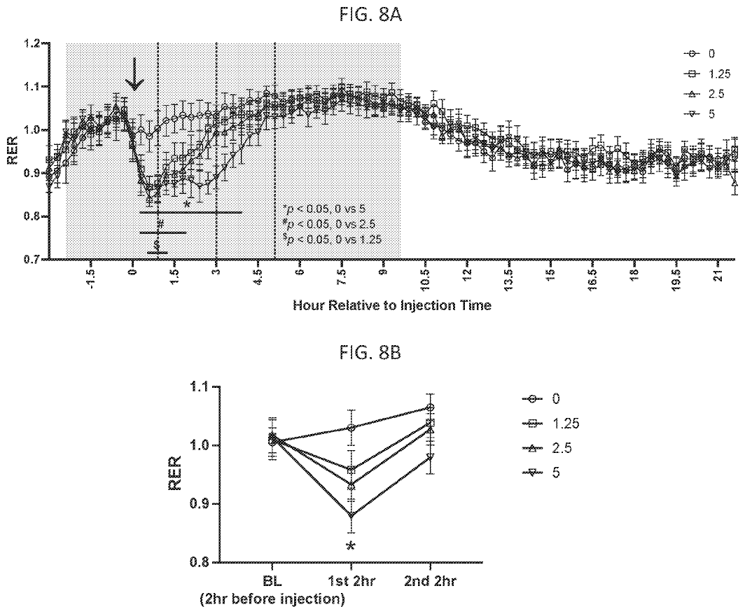

The effect of CYM-5442 on energy metabolism was tested by indirect calorimetry with the Comprehensive Lab Animal Monitoring System (CLAMS; Columbus Instruments, Columbus, OH USA). After 1-week acclimatization, on test day, mice were removed from metabolic chambers, injected with CYM-5442 3 h into the dark phase of the light/dark cycle and then placed back into chambers. After CYM-5442 administration, RER was significantly lower in a dose-dependent manner (FIGS. 8A and 8B). The RER indicates the energy source the organism uses over the day. It is typically measured on a scale from 0.7 to 1. RER value of 0.7 indicates that the organism is burning fat as main fuel, on the contrary, RER value of 1 indicates that the organism is burning carbohydrates as main fuel. These results indicate that CYM-5442 administration induces a shift from carbohydrates to fat as the main source of energy and therefore the ability of CYM-5442 to alter energy homeostasis in mice and increase fat oxidation.

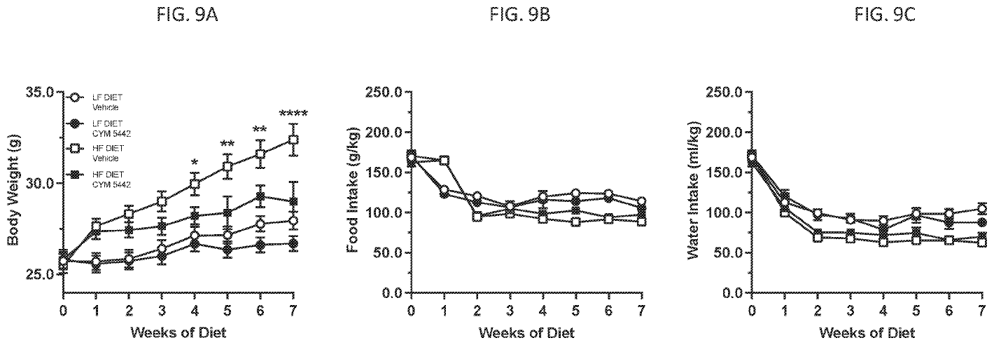

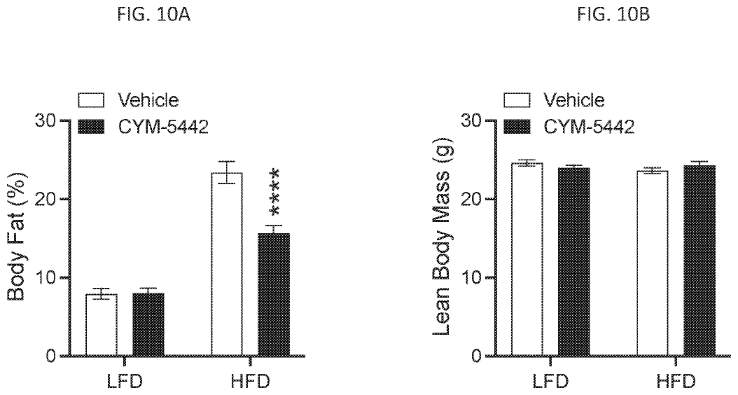

The effect of chronic daily administration of CYM-5442 on body weight and body fat content was tested in mice fed either a low-fat diet (LFD; D12450K, 10 kcal % Fat, Research Diets, Inc. NJ, US) or a high-fat diet (HFD; D07011903, 45 kcal % Fat) ad libitum. Mice were treated with CYM-5442 (2.5 mg/kg, i.p.) or vehicle 5 days a week over 7 consecutive weeks. CYM-5442 effectively prevented body weight gain in HFD-fed mice (FIG. 9A) in comparison to vehicle-treated HFD-fed mice and the LFD-fed groups without overall daily food and water intake (FIGS. 9B and 9C). This is consistent with the moderate half-life of the drug that allows adlibitum fed mice to eat through the day. This finding supports that the metabolic effect of CYM-5442 of increased fat utilization or lipolysis is independent of reduction of food intake. CYM-5442 also reduced body fat content (FIG. 10A) without affecting body lean mass (FIG. 10B).

### Material and Methods

Mice Independent batches of male (n=172) and female (n=165) C57BL/6J mice purchased from The Jackson Laboratories (Bar Harbor, ME, USA) were used. Mice were 6-weeks-old at arrival and single housed in standard plastic cages in a room provided with a reverse 12 h/12 h light/dark cycle (lights off at 9:00 AM), constant temperature (21°±1° C.) and humidity (50%±5%). Food and water were always available except when otherwise reported. Mice were acclimated to housing and colony conditions for 2 weeks before experiment initiation. Behavioral experiments were performed during the dark phase of the light/dark cycle.

### Rats

Male Wistar rats were housed two per cage on a reverse 12 h/12 h light/dark cycle (lights off at 8:00 AM) in a temperature (20-22° C.) and humidity (45-55%) controlled vivarium with ad libitum access to tap water and food pellets (PJ Noyes, Lancaster, NH, USA). Rats were acclimated to housing and colony conditions for 2 weeks before experiment initiation. Behavioral experiments were performed during the dark phase of the light/dark cycle.

All procedures adhered to the National Institutes of Health guidelines for the “Care and Use of Laboratory Animals” and were approved by the Institutional Animal Care and Use Committee of The Scripps Research Institute.

### Drugs

The S1P agonists fingolimod (1, 2, and 4 mg/kg), ozanimod (0.25, 0.5, and 1 mg/kg) and CYM-5442 (1.25, 2.5, and 5 mg/kg) (Tocris, Minneapolis, MN, USA) were suspended in saline with 1% (w/v) Tween 80 and administered intraperitoneally (i.p.) (injection volume in mice 10 ml/kg, in rats 2 ml/kg). Pretreatment time was 30 min for fingolimod and ozanimod, while CYM-5442 was injected 60 min before each behavioral experiment.

### Mouse Experiments

### Drinking the Dark (DID) Paradigm

Acute effect of the S1P agonists was tested in alcohol binge-like drinking in male and female C57BL/6J mice exposed to the drinking in the dark paradigm. As described by Rhodes and colleagues (Rhodes et al. Physiol Behav 84:53-63, 2005; Thiele et al. Alcohol 48:235-41, 2014) 3 h after lights go off, the water bottle of single-housed mice was replaced with a bottle containing 20% (v/v) alcohol and left in place for 2 h, over three consecutive days during which mice stabilized alcohol drinking. On the fourth day, CYM-5442 was injected i.p. 60 min before the start of the drinking session, which had a duration of 4 h, and alcohol intake was measured after the first and second 2 h drinking session. Over the experiment, the amount of alcohol consumed by mice was measured by weighing (0.01 g accuracy) the bottle immediately before and after each drinking session. Alcohol consumption was expressed as grams of pure alcohol per kilogram of body weight (g/kg). In addition, at the end of the second 2 h drinking session, blood samples were collected from the tip of the tail of each mouse and BALs were measured.

An identical experimental plan was adopted to evaluate acute effects of CYM-5442 on the no-drugs reinforcer saccharin (0.002% w/v). Independent groups of C57BL/6J mice were used for testing the effect of CYM-5442 on saccharin intake.

### Blood Alcohol Levels (BALs)

BALs were determined by conventional oxygen-rate alcohol analyzer (Analox Instruments, Atlanta, GA). The reaction is based on alcohol oxidation of alcohol oxidase in the presence of molecular oxygen (ethanol+O₂->acetaldehyde+H₂O₂). The rate of oxygen consumption is directly proportional to the alcohol concentration.

### Food, Water Intake and RER

Effect of CYM-5442 was evaluated on food (g) and water intake (ml) and respiratory exchange ratio (RER: VCO2/VO2) of male C57BL/6J mice (n=30) allocated into CLAMS (Comprehensive Lab Animal Monitoring System, Columbus Instruments, Columbus, OH USA) chambers. After 1-week acclimatization, on test day, mice were removed from the metabolic chambers, injected with CYM-5442 60 min before 3 h into the dark of the light/dark cycle and then placed back into the chambers. Food, water intake and RER were monitored starting the 1^{st} to the 12^{th} h of the dark phase of the light/dark cycle.

### Body Composition

Body composition was determined with a quantitative nuclear magnetic resonance instrument (qNMR, Echo MRI, Houston, TX).

### Rotarod Test

Effect of CYM-5442 on motor coordination was evaluated in male and female C57NBL/6J mice and male Wistar rats in the rotarod apparatus (Rotamex-5, Columbus Instruments, USA). Briefly, one week before testing, animals were extensively habituated to handling and intraperitoneal injections. On test day, animals were weighed and allocated into the four experimental groups. CYM-5442 or vehicle were injected intraperitoneally 60 min before the start of the first trial. Each subject underwent 3 trials of 300 s (intertrial interval 60 s) with a rotarod acceleration from 4 to 40 rpm. Trials in which animals fell before 5 s, calculated from the beginning of the trial, were repeated. For each animal, the latency to fall was assessed and expressed as the average values over the 3 trials.

### Rat Experiments

### Methamphetamine Self-Administration

Male Wistar rats were aseptically implanted with intravenous catheters in the right jugular vein under deep isoflurane anesthesia as previously described in Ahmed et al. (Proc Natl Acad Sci USA 102:11533-8). At the time of testing, the rats' body weights ranged between 350 and 400 g.

Self-administration was performed in operant conditioning chambers (Med Associates, St. Albans, VT, USA) that were enclosed in lit, sound-attenuating, ventilated environmental cubicles. Methamphetamine was delivered through plastic catheter tubing that was connected to an infusion pump. The infusion pump was activated by responses on the right (active) lever. Responses on the left (inactive) lever were recorded but did not have any scheduled consequences. Activation of the pump resulted in the delivery of 0.1 ml of the drug solution. A computer controlled fluid delivery and behavioral data recording. All of the rats were tested for methamphetamine self-administration on a continuous reinforcement (fixed ratio 1, FR1) schedule. Each active lever press resulted in the delivery of one METH dose (0.05 mg/kg/0.1 ml infusion). A 20-s timeout (TO) period followed each METH infusion. During the timeout period, responses on the active lever did not have scheduled consequences. This TO period occurred concurrently with the illumination of a cue light that was located above the active lever to signal delivery of the positive reinforcement. Once lever responding for METH stabilized, rats were divided in two balanced groups and injected either with vehicle or CYM-5442 60 min before the start of the 1 h self-administration session. Subsequently, after a week washout, the effect of CYM-5442 was also tested in rats exposed to progressive ratio (PR) schedule of reinforcement. Under these conditions, the response requirements that were necessary to receive a single drug dose increased according to the following equation: (5e[injection numbers×0.2])−5. This resulted in the following progression of response requirements: 1, 2, 4, 6, 9, 12, 15, 20, 25, 32, 40, 50, 62, 77, 95, 118, 145, 178, 219, 268, etc. The breakpoint was defined as the last ratio that was attained by the rat prior to a 60 min period during which a ratio was not completed.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

What is claimed is:

1. A method of reducing alcohol intake, intake of a drug of abuse, or food intake in a subject comprising administering a pharmaceutical composition comprising a sphingosine-1 phosphate (S1P) receptor (S1PR) modulator in an amount and for a duration sufficient to reduce alcohol intake, intake of a drug of abuse, or food intake.

2. The method of claim 1, wherein the method reduces alcohol intake.

3. The method of claim 1 or 2, wherein the S1PR modulator is an S1PR1, S1PR2, S1PR3, S1PR4, and/or an S1PR5 modulator.

4. The method of any one of claims 1-3, wherein the pharmaceutical composition suppresses dependence or craving for alcohol or a drug of abuse or food in the subject.

5. The method of any one of claims 1-4, wherein the pharmaceutical composition is administered orally.

6. The method of any one of claims 1-5, wherein the subject suffers from or has previously suffered from alcoholism, an alcohol use disorder, alcohol abuse, alcohol dependence, or relapse to excessive drinking.

7. The method of any one of claims 1-6, wherein the subject suffers from or has previously suffered from drug abuse, substance abuse, substance misuse, or relapse to drug taking.

8. The method of any one of claims 1-7, wherein the subject suffers from or has previously suffered from an eating disorder or obesity or is overweight.

9. The method of claim 8, wherein the eating disorder is binge eating disorder, anorexia nervosa, bulimia nervosa, pica, rumination syndrome, avoidant/restrictive food intake disorder, or compulsive eating behavior.

10. The method of any one of claims 1-9, wherein the method results in weight loss, increased fat breakdown or fat oxidation, or fat loss or lipolysis.

11. The method of any one of claims 1-10, wherein the method reduces inflammation.

12. The method of any one of claims 1-11, wherein the S1PR modulator is an agonist.

13. The method of any one of claims 1-12, wherein the agonist is fingolimod, ozanimod, siponimod, ponesimod, A971432, CYM-5442, S1P, TC-SP 14, SEW 2871, RP 001 hydrochloride, CS 2100, RP 001, ASP4058, AMG 369, AUY954, KRP-203, ceralifimod, GSK2018682, CS-0777, VPC22277, carbamoyl-nicotinamide, or a pharmaceutically acceptable salt thereof.

14. The method of claim 13, wherein the pharmaceutical composition comprises fingolimod.

15. The method of claim 13, wherein the pharmaceutical composition comprises ozanimod.

16. The method of claim 13, wherein the pharmaceutical composition comprises CYM-5442.

17. The method of claim 13, wherein the pharmaceutical composition comprises A971432.

18. The method of claim 13, wherein the pharmaceutical composition comprises siponimod.

19. The method of any one of claims 1-11, wherein the S1PR modulator is an antagonist.

20. The method of claim 19, wherein the antagonist is CAY10444, etrasimod, amiselimod (MT-1303), W146, VPC 23019, JTE 013, AB1, CYM 50260, CYM 50308, AAL-R, VPC03090, VPC44116, NIBR-0213, or a pharmaceutically acceptable salt thereof.

21. The method of claim 20, wherein the pharmaceutical composition comprises CAY10444.

22. The method of any one of claims 1-11, wherein the S1PR modulator is a compound selected from Table 1.
